(19) United States
(12) Patent Application Publication
(10) Pub. No.: US 2004/0177400 A1
(43) Pub. Date: Sep. 9, 2004

(54) Methods and constructs for increasing the content of selected amino acids in seeds

(76) Inventors: Tony Mikaer Wahlroos, Turku (FI); Josif Grigorievieh Atabekov, Moscow (RU); Yurii Leonidivich Dorokhov, Moscow (RU); Petri Eerik Susi, Turku (FI); Mauri Jaakko Makela, Turku (FI); Timo Kalevi Korpela, Turku (FI)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/787,393

(22) Filed: Feb. 27, 2004

(30) Foreign Application Priority Data
Feb. 28, 2003 (FI) 2003035

(51) Int. Cl. A01H 1/00; C12N 15/82

(52) U.S. Cl. 800/278

(57) ABSTRACT

The present invention provides constructs and methods for increasing the content of selected amino acids by targeted expression or accumulation of a protein enriched with an amino acid sequence entailing said protein in a plant species or in a tissue or an organ of a plant. The increased content is obtained by stable transformation of plants with a recombinant nucleotide sequence construct encoding a carrier protein having in its 3′-terminal end a polyamino acid extension and operably linked with a tissue or an organ specific regulatory sequence. The increased amino acid content in plant tissues, particularly membraneous oil bodies and cell walls of seed provides a composition useful as animal feed.

### TECHNICAL FIELD OF THE INVENTION

The present invention relates in general to plant biotechnology. Particularly, the invention is related to methods and constructs for increasing the content of selected amino acids in a plant species or in a tissue or an organ of a plant, including cell walls, cell membranes, oil bodies, particularly in seeds. The increased content is obtained by providing a recombinant nucleotide sequence construct encoding a carrier protein having in its 3′-terminal end a polyamino acid extension. Compositions obtainable by the method and the use of said amino acid-enriched composition as well as plants, plant cells and cell-lines transformed with one or more of said constructs are disclosed.

### BACKGROUND OF THE INVENTION

Human beings and livestock require eight essential amino acids in their diets. Diets based predominantly on a single cereal or legume species result in amino acid deficiencies due to nutritional limitation of seed proteins that may have a negative effect(s) on the dietary needs of human beings and animals. For example, the proteins in cereal seeds are deficient in lysine and tryptophan, whereas legume seeds contain proteins deficient in the sulfur-containing amino acids, methionine and cysteine. The use of seed proteins in feed of livestock necessitates that the diet has a prescribed amino acid composition in order to promote the health of animals, efficient growth and good quality of meat and milk. Therefore, it is advantageous to modify existing plant protein resources, in particular, for the composition of essential amino acids in order to be better adapted to the needs of a specified animal.

Efforts have been made to match the composition of vegetable amino acids to the dietary needs of humans and animals, but with limited success. The use of nutritionally superior plant mutants and tissues thereof is however compromised by negative pleiotropic effects. These problems include poor seed germination, slow dry-down, reduced yield, increased microbial and insect susceptibility, and poor milling characteristics.

Genetic engineering provides an alternative means of changing the composition of an essential or any amino acid in plants and tissues thereof. In order to elevate methionine or lysine, either biosynthetic pathways have been manipulated, or high-methionine/lysine proteins have been expressed in transgenic seeds (WO 96/38574; WO 96/01905; WO 95/31554; WO 95/15392; WO 93/19190; EP 485 970, WO 99/40209). Principally, three methods have been applied: 1) the amino acid sequence of endogenous protein(s) was altered; 2) naturally occurring proteins from other plant species were recruited for heterologous expression; and 3) synthetic genes containing high levels of methionine/lysine were expressed.

The method of using high-methionine/lysine proteins from other plants has been disclosed in U.S. Pat. No. 5,633,436, U.S. Pat. No. 5,580,782 and WO 94/16078. With the aim of improving the nutritive value of an important grain legume crop, a chimeric gene specifying seed-specific expression of a sulfur-rich, sunflower seed albumin was stably transformed into narrow-leafed lupin (Lupinus angustifolius L.). Sunflower seed albumin accounted for 5% of extractable seed protein in a line containing a single tandem insert of the transferred DNA. The transgenic seeds contained less sulfate and more total amino acid sulfur than the non-transgenic parent line; this was associated with a 94% increase in methionine content and a 12% reduction in cysteine content. There was no statistically significant change in other amino acids or in total nitrogen or total sulfur content of seeds.

A combination of the first and second approaches (above) has been described in U.S. Pat. No. 5,850,016. To improve the methionine content of potato tubers, a cDNA clone encoding Brazil nut 2S albumin was mutagenized to increase its methionine content by 2-7 additional methionine residues and transformed into potato plants. Irrespective of the mutation, protein content in leaves was low, ranging from <0.01%-0.2% of total protein.

The use of synthetic proteins has been disclosed in FR 2,744,134, U.S. Pat. No. 5,559,223 and WO 92/14822. To increase the lysine and methionine content in seeds, a synthetic protein based on an α-helical coiled-coil structure containing 31% lysine and 20% methionine (CP3-5) was designed. Driven either by the phaseolin or β-conglycinin promoter, moderate amounts of the synthetic protein accumulated in seeds harvested from transgenic tobacco plants.

In WO 99/15004 a chimeric construct for modifying the composition of storage organs in plants is described. A gene encoding a sulphur-rich protein is provided with a C-terminal KDEL extension, which enables targeting of the construct to the endoplasmic reticulum and Golgi apparatus.

The above approaches have certain drawbacks. When large amounts of foreign proteins with no functional role for the host plant are expressed it may result in many secondary problems connected with physiological abnormalities of seeds as noticed in equivalent-type mutants created by traditional breeding methods.

The first objective of the present invention is to provide a method, including recombinant nucleotide sequence constructs, enabling selection of constructs for effective transformation of any desired plant species with targeted expression enabling accumulation of stable protein enriched by one or more selected amino acids entailing said protein i n any selected plant tissues. When using said constructs the detrimental effects of expressing large amounts of foreign proteins with no functional role for the host plant can be avoided. A second objective of the present invention is to provide a composition comprising a stable amino acid-enriched protein, which has accumulated in selected plant tissue combined with compatible formulation aiding additives. The use of said composition to be suitable as a direct food source from selected plant for humans as well as fodder, especially as animal feed and as a feed supplement, is suggested.

### SUMMARY OF THE INVENTION

The present invention is related to improving the quality of plant proteins by a method, which increases the content of one or more selected amino acids by targeted expression or accumulation of a protein enriched with an amino acid sequence entailing said plant protein. The method comprises the steps of transforming a plant with at least one recombinant nucleotide sequence construct. The construct comprises tissue or organ specific regulatory sequences, which drive transcription during selected stages of morphogenesis. The regulatory sequences are operably linked to a chimeric nucleotide sequence. The chimeric nucleotide sequence, which is used for transforming plants, particularly crop plants comprises two key elements—a nucleotide sequence encoding a carrier protein with intact functional properties as compared to a native carrier protein and a nucleotide sequence comprising codons encoding an amino acid sequence having four to eighty amino acid residues and comprising a combination of one ore more selected amino acid entailing the functionally intact carrier protein. The nucleotide sequence encoding the carrier protein is selected from nucleotide sequences encoding plant specific proteins enabling targeted expression or accumulation of the amino acid-enriched protein in a selected tissue or organ of the plant.

The nucleotide sequence encoding the carrier protein lacks a termination codon and is from its 3′-terminal end fused in-frame with nucleotide sequences comprising a selected number of codons encoding a selected combination of one or more amino acid residues. Said construct enables stable targeted expression or accumulation of the amino acid-enriched carrier protein having a polyamino acid extension in the selected plant tissue or organ.

The above described so called transformation construct is obtained by selecting constructs enabling stable protein translation of the polyamino acid extension and stable targeted expression or accumulation. The selection is carried out with an in vitro translation system (IVT) and/or a transient expression system and is subsequently confirmed with other applicable methods.

The IVT system is primarily used to determine the optimal number of codons, which can be fused in-frame with the nucleotide sequence encoding the carrier protein without comprising the efficacy of the translation of the amino acid-enriched entailed carrier protein.

The transient expression system comprises the construct described above fused in-frame with a nucleotide sequence encoding a reporter protein. This transient expression construct is introduced into a plant cell, preferably using a microprojectile bombardment method. Thereafter, constructs providing stable, targeted expression of the reporter gene in said plant cell are selected and the nucleotide sequences encoding the reporter protein from the selected constructs are removed. Said constructs are used for production purposes by transforming plants with the constructs lacking the reporter gene as described above, preferably using Agrobacterium-mediated transformation systems.

The method allows production of amino acid-enriched proteins, i.e. carrier proteins entailed with a polyamino acid extension comprising a selected combination of four to eighty amino acids residues. The amino acids are advantageously histidine, cysteine, methionine, glycine, lysine, tryptophan, alanine, valine, leucine, isoleucine, proline, phenylalanine, tyrosine, serine, threonine, arginine, aspartate, glutamate, asparagines, glutamine or any combination thereof.

The carrier protein enabling stable targeted expression or accumulation in a tissue or organ of a plant may be selected from proteins functioning in the intracellular trafficking pathway of the plant. Advantageously the carrier protein is a cell wall protein or plant viral protein. Useful carrier proteins are for example oleosin, caleosin, steroleosin, cruciferin, napin or a plant viral movement protein.

The regulatory sequence is favourably a promoter expressing during embryogenesis. Useful regulatory sequences comprise promoters, such as the napin (NAP), 35S, chimeric hybrid (HYB), 19S, nopalin, phaseolin, steroleosin, caleosin, cruciferin, Alfalfa mosaic virus (AMV), heat-shock, albumin 2S or oleosin promoters.

The reporter protein is advantageously a detectable protein. Fluorescent proteins, such as green fluorescent protein (GFP), a red fluorescent protein, a β-glucuronidase, an obelin or a luciferase are particularly advantageous.

The method of the present invention is particularly useful for producing a composition comprising in plant material, including cell wall debris, an amino acid-enriched carrier protein having a polyamino acid extension, wherein the content of selected amino acids in the plant material obtained by the method as compared to the amino acid content in a corresponding unmodified wild type plant is at least 2:1. Said composition is useful for for producing an amino acid-enriched feed of an oil cake obtained after recovery of oil from plants.

Also disclosed in the present invention are plants, plant cells and plant cell lines transformed with the transient expression constructs or transformation constructs.

The characteristic features of the present invention are as defined in the claims.

### A BRIEF DESCRIPTION OF THE DRAWINGS

FIG. 1 shows eighteen expression plasmids, wherein pRT means a construct containing a 35S promoter of Cauliflower mosaic virus in a plasmid vector pRT100 (Töpfer et al., Nucleic Acid Res. 15(14): 5890, 1987). The eighteen plasmids have been constructed using genes of either of two carrier proteins, oleosin (OLE) or Tobacco mosaic virus movement protein (MP) with one of three transcriptional promoters indicated by arrows (NAP=napin promoter, 35S=Cauliflower mosaic virus 35S promoter, HYB=hybrid promoter i.e. the enhancer region from the CaMV 35S promoter fused to the napin promoter) and internal histidine (His) or cysteine-methionine (Cys-Met) codon-enriched nucleotide sequence regions (“cassettes”; see FIG. 3 for cassette details) of varying length (2× contains two cassettes, 4× contains four cassettes, and 6× contains six cassettes) depicted with grey boxes. The green fluorescent protein (GFP) coding region was fused in-frame to sequences enriched with His or Cys-Met codons. Arrows represent promoters, and the small box labelled as “t” indicates the transcription termination sequence. Location of several restriction sites are indicated in the Figure.

FIG. 2 shows four expression plasmids that were selected on the basis of transient expression assay data for stable transformation into Brassica campestris plants. NAP=napin promoter, HYB=hybrid promoter i.e. the enhancer region from the CaMV 35S promoter fused to the napin promoter, OLE=oleosin, MP=Tobacco mosaic virus 30K movement protein gene, GFP=green fluorescent protein gene, 4x= four amino acid-enriched nucleotide sequence regions (cassettes). Arrows represent promoters, and the small box labelled as “t” indicates the transcription termination sequence. Location of several restriction sites are indicated in the Figure. FIG. 3 shows the nucleotide sequence of a histidine codon-enriched sequence (A), the nucleotide sequence of a cysteine-methionine codon enriched sequence (B), the nucleotide sequence of a glycine codon-enriched sequence (C) and the nucleotide sequence of a lysine codon-enriched sequence (D). The protein translation sequence is shown below the nucleotide sequence. K, lysine; H, histidine; R, arginine; V, valine; G, glycine; L, leucine; C, cystine; S, serine; M, methionine. The asterisk indicates the transcriptional termination codon. Restriction sites used in cloning are shown in bold and are indicated above the sequence.

FIG. 4 shows the construction scheme of plasmids carrying fusions of histidine or cysteine-methionine codon-enriched DNA sequences of different lengths fused to the TMV 30K MP gene (open box “MP”). pGEM-7Zf(+) (Promega Corporation, U.S.A; catalogue number P2251) was used to construct histidine codon-enriched nucleotide sequences. Clone pGEM-His-24 contains a DNA segment coding for a 19 amino acid-long peptide containing 14 His residues (FIG. 3A). The sequence of this DNA fragment was flanked by XhoI and BamHI sites and contained also a SalI restriction site designed for subsequent cloning steps (FIG. 3A). Histidine or cysteine-methionine codon-enriched DNA sequences are depicted by gray boxes of different lengths. pGEM-His-24 is used as an example for a fusion of histidine-enriched DNA sequence to the TMV 30K MP gene (Example 11). The same construction scheme can be applied for carrying a fusion of cysteine-methionine codon-enriched DNA sequences to the TMV 30K MP gene (Example 12). Location of several restriction sites are indicated in the Figure.

FIG. 5A depicts the construction of expression plasmids pNAP and pHYB, wherein RT means that the construct contains the 35S promoter of Cauliflower mosaic virus (CaMV 35S promoter) from a plasmid vector pRT100 (Töpfer et al., Nucleic Acid Res. 15(14): 5890, 1987). The expression plasmids contain the napin promoter (NAP) or chimeric promoter (HYB) that consists of the entire napin promoter coupled with the enhancer region of the Cauliflower mosaic virus (CaMV) 35S promoter (Example 16). Arrows represent promoters, and the small box labelled as “t” indicates the transcription termination sequence. Location of several restriction sites are indicated in the Figure.

FIG. 5B depicts the cloning of the green fluorescent protein (GFP) gene into 35S CaMV and napin promoter-based plasmids, wherein RT means that the construct contains the 35S promoter of Cauliflower mosaic virus (CaMV 35S promoter) from a plasmid vector pRT100 (Töpfer et al., Nucleic Acid Res. 15(14): 5890, 1987). The expression plasmids contain the napin promoter (NAP) or chimeric promoter (HYB) that consists of the entire napin promoter coupled with the enhancer region of the Cauliflower mosaic virus (CaMV) 35S promoter (Example 16). Arrows represent promoters, and the small box labelled as “t” indicates the transcription termination sequence. Location of several restriction sites are indicated in the Figure.

FIG. 6 shows the relative GFP expression achieved using the 35S CaMV (a) and HYB (b) promoters in constructs pRT-OLE-4x-GFP and pHYB-OLE-4x-GFP, respectively (see FIG. 1) after particle bombardment of constructs into epidermal cells of Nicoliana benthamiana (Example 8).

FIG. 7 depicts the construction of plant expression vectors consisting of the TMV 30K MP gene (MP), His- or Cys-Met-enriched sequence cassettes of different length (grey boxes) fused to the GFP coding region, under the control of the 35S CaMV promoter. RT means that the construct contains the 35S promoter of Cauliflower mosaic virus (CaMV 35S promoter) from a plasmid vector pRT101 (Töpfer et al., Nucleic Acid Res. 15(14): 5890, 1987). Arrows represent promoters, and the small box labelled as “t” indicates the transcription termination sequence. Location of several restriction sites are indicated in the Figure.

FIG. 8 depicts the construction of plant expression vectors consisting of the TMV 30K MP gene (MP), His- or Cys-Met-enriched sequence cassettes of different length (grey boxes), fused to the GFP coding region, under the control of the napin (NAP) promoter. RT means that the construct contains the 35S promoter of Cauliflower mosaic virus (CaMV 35S promoter) from a plasmid vector pRT100 (Töpfer et al., Nucleic Acid Res. 15(14): 5890, 1987). pGEM-30K contains the 30K movement protein gene from TMV in pGEM-7Z(+) plasmid (Promega Corporation USA) (Example 7). Arrows represent promoters, and the small box labelled as “t” indicates the transcription termination sequence. Location of several restriction sites are indicated in the Figure.

FIG. 9 depicts the construction of plant expression vectors consisting of the TMV 30K MP gene (MP), His- or Cys-Met-enriched sequence cassettes of different length (grey boxes), fused to the GFP coding region, under the control of the hybrid (HYB) promoter. RT means that the construct contains the 35S promoter of Cauliflower mosaic virus (CaMV 35S promoter) from a plasmid vector pRT100 (Töpfer et al., Nucleic Acid Res. 15(14): 5890, 1987). pGEM-30K contains the 30K movement protein gene form TMV in pGEM-7Z(+) plasmid (Promega Corporation USA) (Example 7). Arrows represent promoters, and the small box labelled as “t” indicates the transcription termination sequence. Location of several restriction sites are indicated in the Figure.

FIG. 10 depicts the construction of plant expression vectors (pRT) consisting of oleosin gene (OLE), His- or Cys-Met-enriched sequence cassettes of different length (grey boxes), fused to the GFP coding region, under the control of the 35S CaMV promoter. RT means that the construct contains the 35S promoter of Cauliflower mosaic virus (CaMV 35S promoter) from a plasmid vector pRT100 (Töpfer et al., Nucleic Acid Res. 15(14): 5890, 1987). See Example 6 for details of pOLE4/11. Arrows represent promoters, and the small box labelled as “t” indicates the transcription termination sequence. Location of several restriction sites are indicated in the Figure.

FIG. 11 depicts the construction of plant expression vectors consisting of oleosin gene (OLE), His- or Cys-Met-enriched sequence cassettes of different length (grey boxes), fused to the GFP coding region, under the control of the napin (NAP) promoter. See Example 6 for details of pOLE4/11. Arrows represent promoters, and the small box labelled as “t” indicates the transcription termination sequence. Location of several restriction sites are indicated in the Figure.

FIG. 12 depicts the construction of plant expression vectors consisting of oleosin gene (OLE), His- or Cys-Met-enriched sequence cassettes of different length (grey boxes), fused to the GFP coding region, under the control of the hybrid (HYB) promoter. See Example 6 for details of pOLE4/11. Arrows represent promoters, and the small box labelled as “t” indicates the transcription termination sequence. Location of several restriction sites are indicated in the Figure.

FIG. 13 shows a Western blot of pNAP-MP-4xHis-GFP (see FIG. 2) expressing Brassica campestris plants. Lanes 1, 2 and 3 correspond to three independent pNAP-MP4xHis-GFP transformants (lines 5.1A7, 5.1A11 and 5.1A18, respectively). Molecular weight markers (dots on the right hand side) are 50 kDa (upper dot) and 40 kDa (lower dot). His-antibody was used as the probe.

FIG. 14 shows a Western blot of pHYB-OLE-4xHis-GFP (see FIG. 2) expressing Brassica campestris plants. Lanes 1, 2, 3, 5, 6 and 7 correspond to six independent pHYB-OLE-4xHis-GFP transformants (lines 17.1237, 17.1238, 17.1240, 17.20c8, 17.20c11 and 17.20c20, respectively); lane 4 corresponds to a wild-type (untransformed) control plant. Molecular weight markers (lane 8, dots on the right hand side) are 50 kDa (upper dot) and 40 kDa (lower dot). His-antibody was used as the probe.

FIG. 15 shows a Western blot of pNAP-OLE-4xHis-GFP (see FIG. 2) expressing Brassica campestris plants. Lanes 2, 3, 5, 6, 7 and 8 correspond to six independent pNAP-OLE-4xHis-GFP transformants; lane 4 corresponds to a wild-type (untransformed) control plant. Molecular weight markers (lanes 1 and 9) are 50 kDa (upper dot) and 40 kDa (lower dot). His-antibody was used as the probe.

### DETAILED DESCRIPTION OF THE INVENTION

### Definitions

The terms used in the present invention have the meaning they usually have in the fields of plant biotechnology, protein chemistry and feed formulation. Some terms in the present invention are, however, used in a broader or somewhat different manner. Therefore, some of the terms are defined in more detail below.

“Recombinant nucleotide sequence construct” or simply “construct” means a DNA construct, a transient expression cassette or vector or a transformation cassette or vector. The construct may comprise linear or circular end-to-end linked nucleotide sequences optionally inserted in a plasmid. In order to prepare effective, stable transformation constructs, transient or intermediate constructs expressing a reporter protein are designed and stable well performing transient or intermediate constructs are identified and selected to provide effective, stable transformation constructs for production purposes. From said selected, effective constructs the reporter sequence is subsequently removed to provide transformation constructs for transforming the selected plant species, which are finally used for production purposes. In the present invention the transformation construct comprises regulatory sequences and a nucleotide sequence encoding a carrier protein fused in-frame with codons encoding the selected amino acid residues. The transient construct further comprises a nucleotide sequence encoding a detectable reporter protein. In the transient or intermediate construct at least one nucleotide sequence cassette is included, but preferably more, for example two up to six cassettes. Each cassette comprises at least two, preferably five or more codons encoding the selected amino acid residues. Cassettes comprising up to ten or fifteen of the selected amino acid codons may be fused between the nucleotide sequences encoding the carrier and the reporter protein. It is to be noted that nucleotide sequences encoding the reporter protein are removed from constructs, which have been found to be stable and effective, i.e. they perform as desired by showing targeted expression. These inserted constructs lacking a reporter gene areused as transformation constructs. Usually the amount of amino acid codons in a stable construct are from four to eighty.

“A cassette of nucleotide sequences” or “a nucleotide sequence cassette” comprises a set of codons, which encode a polyamino acid sequence, the so called “amino acid cassette”. In the present invention cassette means an insert comprising a continuous nucleotide sequence having at least two, preferably four and any number up to about eighty codons or triplets. The cassettes are particularly convenient when designing different constructs and for checking their properties especially their performance in translation. However, the cassettes are not a prerequisite in the present invention. The ultimate goal is to provide a construct that encodes a carrier protein, which has a polyamino acid extension, but still has the same functionally intact properties as the corresponding native carrier protein. Preferably, the optimal number of codons is one that provides a stable carrier protein entailed with the polyamino acid extension and thereby enriched with the selected amino acid residues. Typically the number of amino acid residues, which can be stably attached as an extension is from four to about eighty amino acid residues or any number therebetween. The term “amino acid cassette” is also used for a nucleotide sequence or codons encoding the polyamino acid chain, which may comprise a high amount of one or more selected amino acids and which chain is stably attached to the carrier protein. The number of amino acid residues, which can be attached to the carrier protein, may be determined by randomly inserting nucleotide sequence cassettes comprising different amounts of amino acid codons into the construct and by screening in a cell free translation system to confirm correct codon translation.

The selection of the “stable constructs” is conveniently performed with a “cell free translation system”, which means an in vitro translation (IVT) system in which a normal cellular reaction is reconstituted in the absence of cells, including, for example IVT systems that can synthesise protein from mRNA using e.g. a lysate of rabbit reticulocytes or wheat germ. A particularly advantageous system is the wheat germ cell-free translation system available for example as (TNT® T7/SP6 Coupled Wheat Germ Extract System L5030, Promega corporation).

“Targeted expression or accumulation” means that the amino acid-enriched protein is expressed in specific, selected plant tissues or organs or is transported to said selected tissues or organs. This can be achieved by selecting carrier proteins, which take part in “intracellular trafficking pathways”. Such pathways are the membrane component-specific trafficking pathway, the organelle-specific trafficking pathway, etc. The intracellular trafficking pathways in the plant transport the expressed protein to the selected organs and tissues and thereby enable accumulation of the expressed product for example in the cell walls or cell membranes in seed. When the expression of amino acid-enriched carrier proteins in seeds of transgenic Brassica species is targeted to the cell walls of the seed cells, it enables the anchoring of the amino-enriched protein to the oilcake remaining after pressing out the oil. The oil cake, provided with improved properties, i.e. an increased amino acid content, is a useful ingredient in composition for the feed industry. In a similar manner, the amino acid-enriched protein can be expressed in mesophyll tissue of lettuce, and used directly as human nutrient source.

“The selected amino acid” may be any amino acid, but preferably the amino acid is one or more of the eight essential amino acids required by human beings or livestock. The selected amino acids may be, for example histidine, cysteine, methionine, glycine, tryptophan, lysine, alanine, valine, leucine, isoleucine, proline, phenylalanine, tyrosine, serine, threonine, arginine, aspartate, glutamate, asparagine or glutamine. Any amino acid-enriched protein may be prepared by method of the present invention, particularly as exemplified by the preparation of a histidine-enriched protein. The amino acid-enriched protein is obtainable by providing a construct comprising a nucleotide sequence encoding a carrier protein with an extension with an optimal number of one or more codon cassettes encoding the desired amino acids e.g. His, Met-Cys, His-Met-Cys, etc. In the preferred intermediate or transient construct of the present invention the amino acid codons are fused (situated or placed) between the nucleotide sequences encoding the carrier and the reporter protein in such a way that the additional amino acid residues do not disturb the normal biological functions of the carrier protein, by preventing it from taking part in a secretory intracellular trafficking pathway.

“Carrier protein” in the present invention means a protein, which can be stably extended by a polyamino acid sequence or a peptide. The nuclotide sequence encoding the functionally intact carrier protein is fused in-frame with a nucleotide cassette comprising one or more selected codons encoding amino acid residues. The inserted codons encoding the desired amino acid residues may not disturb the normal biological functions of the carrier protein as compared with the corresponding native unmodified protein. Nucleotide sequences encoding carrier proteins useful in the present invention are selected from plant specific proteins. These plant specific proteins use secretory intracellular trafficking pathways, which enable accumulation of the amino acid-enriched protein in cell walls or membranes. Particularly useful are oil body membranes, plasma membranes, vacuole membranes, plastid membranes of selected plant tissues or organs, including seeds, leaves, roots and tubers etc. In the present invention carrier proteins have been derived from genes encoding three major seed proteins. Said genes are useful model proteins for carrying out genetic engineering with seed proteins, particularly seed proteins from the family Cruciferae. Yet, similar genetic engineering can be applied in any other plant species. Said three exemplified proteins, which are applied in the present invention are the seed proteins cruciferin (500 amino acid residues), napin (165 amino acids) and oleosin (165 amino acids), but other proteins, such as caleosin and steroleosin can be used as well. All the above mentioned proteins are present in native seeds of B. campestris and are poor in histidine content. Cruciferin contains only 9 His residues per 500 amino acids. Napin contains only 2 His residues per 165 amino acids and oleosin does not contain His at all. Cruciferin, oleosin, napin, caleosin and steroleosin are applicable to be used in seeds for feed applications.

In the present invention oleosin (OLE) has been used as a model carrier protein, but other carrier proteins can be used in a similar manner. For example, two related proteins, caleosin and steroleosin mentioned above are suggested as potentially useful due to the similar way they accumulate in oil bodies and cell walls of seeds. Oleosin is reviewed e.g. in (i) Murphy 1996. TIBTECH 14. 206-213; (ii) Methods in Mol.Biol. vol. 44: A grobacterium protocol. Eds. K. M. A. Gartland and (iii) M. R. Daey, Humamana Press Inc. Totowa, N.J.; and Brassica Oilseeds: Production and Utilization. Eds D. S. Kimber and D. I. McGregor. Cab International. 1995. Oleosin was shown to be a useful carrier protein, because it is a hydrophobic protein, has a relatively small size and is a component of the membranes surrounding the storage oil-bodies of B. campestris seeds.

When transferring a nucleotide sequence derived from a selected plant to another selected plant, there is a potential risk of post-transscriptional gene silencing, especially, if the plants are closely related or highly homologous. The risk of inducing post-transcriptional gene silencing in a transgenic plant due to nucleotide sequence homology between the transgene and the endogenous gene may be avoided by using nucleotide sequences encoding carrier proteins having as little homology as possibly. For example in the present invention a carrier protein (encoded by Ole gene) from Arabidopsis thaliana, and not that from B. campestris was successfully used to avoid the problem when transforming B. campestris. An advantage of using a gene encoding a movement protein (MP) is that this gene has no sequence homology to endogenous genes of B. campestris.

The “carrier proteins” of the present invention include isomers, amino acid sequences with minor modification in some amino acid residues. The carrier protein may be a shortened form of tha native carrier protein. The nucleotide sequences encoding the “carrier protein” may also vary to a certain degree. They may for example be truncated. The only prerequisite is that the gene encoding the carrier protein has intact functional properties or biological functions which are substantially the same as those of the native carrier protein, meaning targeted expression in selected plant tissues or organs.

The “intact functional properties” means that the expresion of said carrier protein may be targeted into a selected tissue or organ or compartment of the plant. In other words the carrier protein with the polyamino acid extension must accumulate in the selected tissue, organ or compartment of the plant

“Regulatory sequence” means nucleotide sequences, which regulate the transcription and expression of the structural nucleotide sequences either by down-regulating or up-regulating transcription and expression. Regulatory sequences comprise promoters, enhancers, signal sequences, terminators, etc. The preferred promoters are relatively short, organ or tissue specific transcriptional promoters capable of driving the transcription of the chimeric nucleotide sequence during different stages of morphogenesis and particularly during embryogenesis. Other potentially useful promoters are plant virus-derived promoters from Alfalfa mosaic virus (AMV) or Cauliflower mosaic virus (CaMV) 19S, and those that can be regulated either by environmental effects (e.g. heat) or under specific abiotic (e.g. salicylic acid-responsive) or biotic (pathogen) stress conditions.

The preferred and exemplified “transcription promoters” in the present invention are a napin (NAP) promoter, particularly one from Arabidopsis thaliana, a 35S CaMV promoter or a chimeric “hybrid” (HYB) promoter, which comprises the entire napin promoter coupled with the enhancer sequence of the CaMV 35S promoter. Naturally, promoters can include any other operating regulatory sequences, particularly promoters enabling accumulation of amino acid-enriched proteins in selected tissues. Preferred tissues are oil bodies and cell walls of seeds.

The napin promoter controlling the expression of the napin (NAP) gene is regulated during embryogenesis and is switched on after flowering. The nucleotide sequence of NAP promoter from A. thaliana has been reported (Rask et al. 1998. J. Plant. Physiol. 152, 595-599). An important advantage of the napin (NAP) promoter in the present invention is that it is rather short (152 bp) in comparison to the promoter of the Ole gene which is 2.1 kb.

The 35S promoter of Cauliflower mosaic virus (CaMV) has been shown to be active in cells of B. campestris (Harpster et al., 1988. Mol. Gen. Genet. 212, 182-190). The 35S promoter is also active in embryos and the detection of transgenic B. campestris plants would be facilitated by testing the product of amino acid-enriched recombinant protein (OLE or MP) in mature leaves before flowering.

A “reporter sequence” or “nucleotide sequence encoding a reporter protein” means a nucleotide sequence used to design an intermediate or transient construct, which enables the selection of such constructs, which allow stable targeted expression or accumulation of stable amino acid-enriched proteins with intact biological functions in the selected tissues or organs of the plants. In this case, the nucleotide sequence encoding the reporter protein enables easy, accurate and unambiguous identification of suitable constructs. The reporter sequence enables demonstration of the tissues or organs in which the promoter is activated and the conditions under which the promoter is active. In the preferred embodiments of the present invention the reporter protein is a visible or detectable protein, preferably a fluorescent protein. Preferably it is a green fluorescent protein, β-glucuronidase, obelin or luciferase. The construct with said reporter sequence is an intermediate or transient construct because the reporter sequence can be removed as soon as it has been demonstrated that carrier proteins comprising selected polyamino acid extenion is appropriately expressed. Said reporter genes are removed because they may not be present in the actual transformation constructs, which are used for production of feed or food products.

In the present invention two different systems were used to expedite the selection of useful, stable constructs. A cell-free in vitro translation (IVT) system and a transient expression system were used to select suitable transformation constructs, which allowed undisturbed expression of the carrier protein and the amino acid cassette. The cell-free in vitro translation (IVT) system enabled the identification and thereby the selection of the optimal number of codons that could be translated correctly without any problems.

A transient expression assay was used to ensure protein expression and that the expressed protein had intact biological function(s) as compared to the native unmodified protein, and was expressed and accumulating in the targeted tissue or organ of the plant. This was facilitated by visual observation of the reporter protein. Confocal laser scanning microscopy was particularly useful because it allowed early detection of transgenic plants expressing amino acid-enriched proteins in-frame with the nucleotide sequence encoding the reporter protein. Antibody-based assays (e.g. enzyme linked immuno absorbent assays) and direct amino acid analysis could be used as alternative systems to detect transgenic plants expressing amino acid-enriched proteins in-frame with the nucleotide sequence encoding the reporter protein.

### General Description of the Invention

The primary objective of the present invention was to elaborate methods providing a means to increase the content of selected amino acids in plant cells tissues, such as oil bodies and cell walls, particularly, in seeds of the family Cruciferae. With the method of the present invention, stable amino acid-enriched protein was successfully expressed during embryogenesis of transgenic Brassica campestris plants and the expressed amino acid-enriched protein accumulated in the seeds. With the method and constructs of the present invention the said recombinant amino acid-enriched protein was tightly bound in seed cells to membranes or cell walls (CW) and, therefore, it could be retained in seed remnants, anchored to the oilcake, after the oil was pressed out. This enabled oil to be recovered with subsequent recovery and use of the remains, i.e. the oil cake, which with its improved property, i.e. increased amino acid content, provides a useful ingredient in an amino acid-enriched composition for animal feed preparation. When the extra amino acid, for example, histidine, is in cow fodder, and is bound into normal, native proteins as in the present invention, it functions as a natural amino acid source and can be utilized normally in cow metabolism. Therefore, an amino acid-enriched, for example histidine-enriched, protein is a useful ingredient in fodder for cows functioning as a booster of milk production. Enrichment of other useful amino acids, for example, in edible lettuce leaves would allow the use of such amino acid-enriched protein(s) as a direct human food source with no further processing.

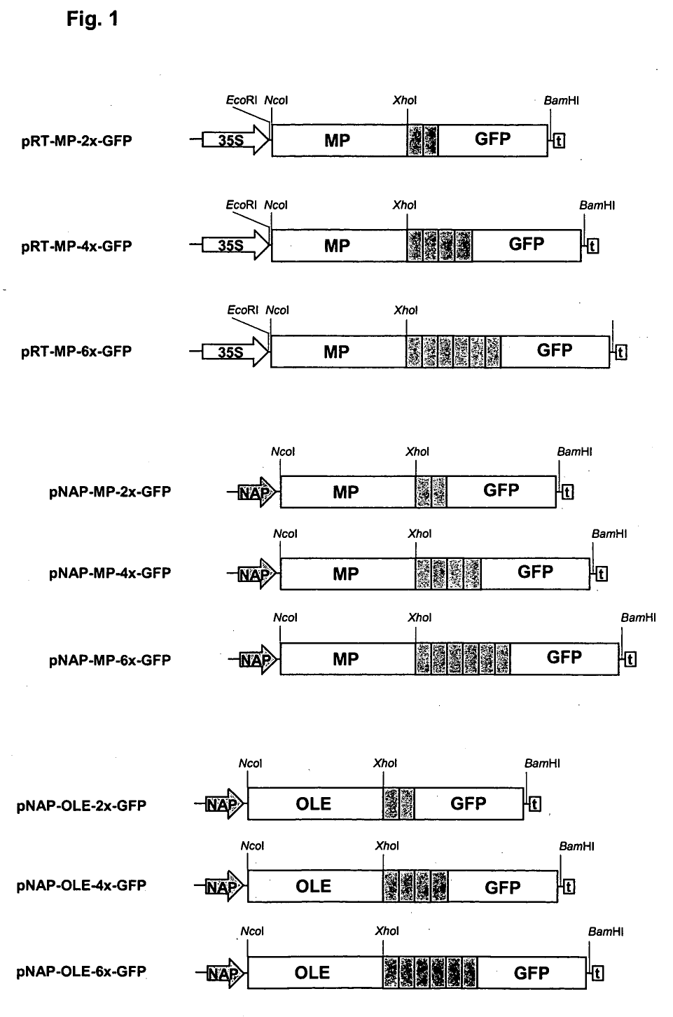

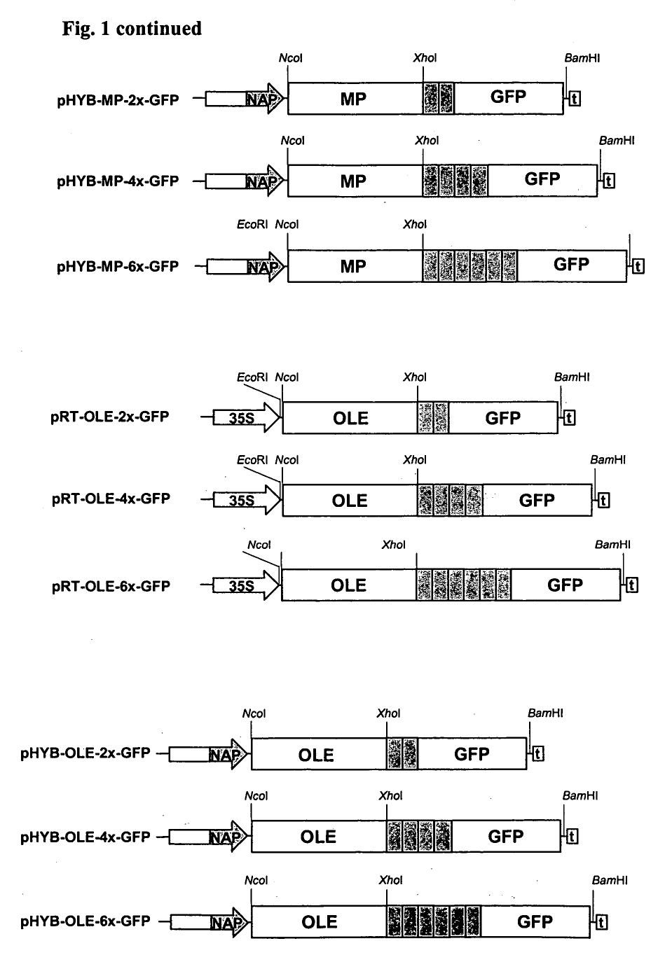

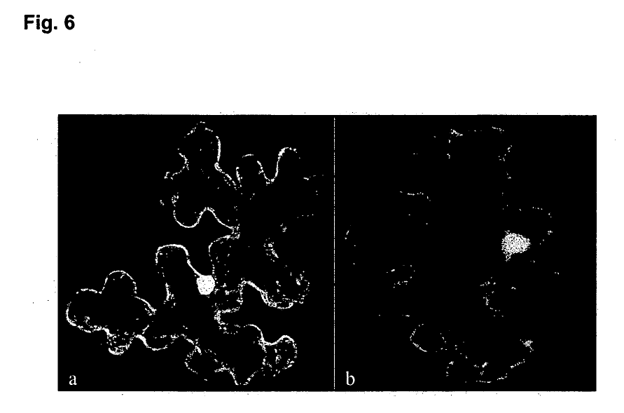

Nucleotide sequences encoding reporter-proteins, exemplified by the visually detectable green fluorescent protein (GFP), positioned downstream of the selected, desired amino acid-codon enriched sequence(s) allowed easy detection of gene expression and intracellular localization of the expressed proteins in transgenic plants. Expression of reporter protein, from various reporter fusion constructs is explified with GFP and illustrated in FIG. 1 and shown in FIG. 6, which shows transient expression of the selected reporter fusion constructs in single cells, exemplified by epidermal tobacco cells.

Therefore, the present invention pertains to methods for producing transformed plants, plant cells, or cell-lines that are capable of expressing a high level of amino acid-enriched carrier proteins having a stable polyamino acid extension, which are in particular localized in selected plant tissues or plant organs or plant cell compartments including seed membraneous oil bodies or cell walls.

The method of the present invention comprises transformation of plants or plant cells with constructs enabling expression of carrier proteins entailed with an polyamino acid sequence or extension in a targeted tissue or organ of the plant The construct for stable transformation comprises regulatory sequences including an organ—and/or tissue-specific transcriptional promoter driving the transcription of the gene(s) of interest, encoding the carrier proteins during different stages of morphogenesis, particularly during embryogenesis. Said regulatory sequences are operably linked to: (a) a nucleotide sequence encoding a carrier protein without a termination codon; and (b) a nucleotide sequence comprising at least one cassette including at least two codons encoding the desired amino acid residues fused in-frame with the nucleotide sequence encoding the carrier protein.

Various constructs may be made with or without the reporter gene. Constructs encoding amino acids under the control of the regulatory sequences including selected promoters may be analysed using in vitro translation (IVT) systems, which allows observations of the stability of the plasmids possessing of different amino acid cassettes. Reporter constructs may also be analysed using transient assays.

Stable constructs comprising the nucleotide sequences encoding the carrier protein entailed at its 3′-terminal end with the amino acid cassettes were selected using the IVT-system by providing constructs with a randomly selected number of amino acid codons, e.g. at least two amino acid codons, inserted as at least one cassette, but preferably more, for example two, four, six or eight cassettes and analyze the results obtained. If the cassette, for example, comprises 14 amino acid codons (14×), this results in amino acid-enriched carrier proteins having 28(2×), 56(4×) and 112 (8×) amino acid-codons downstream of the gene encoding the functionally intact carrier protein (Ole or MP). Preliminary tests indicated that 8× (112 amino acids) were unstable producing deletion variants (mini-plasmids). Also some of the 6× (84 aa) clones were unstable. Thus, for further work clones 2×, 4× and 6× were fused with the 3′-proximal reporter gene, such as the GFP gene with 3′-termination sequences. In precisely the same way it is possible to determine the optimal number of any amino acid codons, by inserting varying numbers of amino acid codons into the construct comprising the nucleotide sequence encoding the carrier protein and select constructs providing stable results.

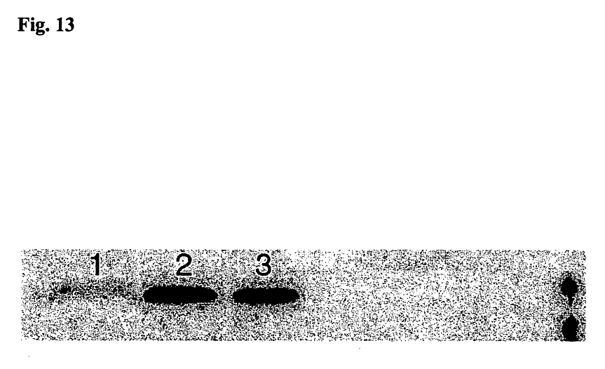

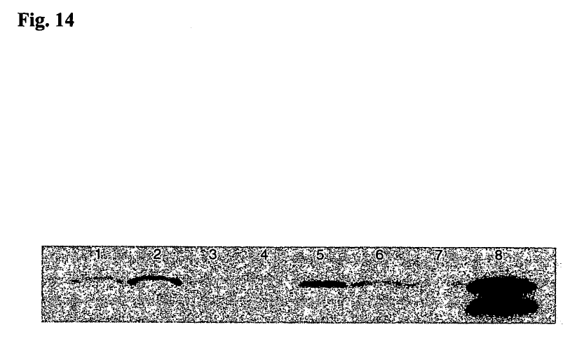

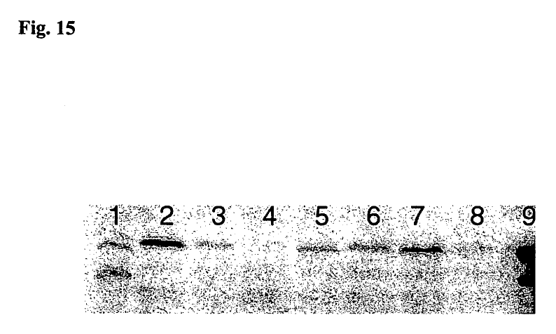

Western blot analysis of several plant lines (Table 1) showed that antibodies raised against all kinds of carrier proteins, amino acids, or reporter proteins, exemplified by histidine sequences, MP, oleosin or GFP (FIGS. 13-15) could be used to detect proteins with known molecular weights (MWs). The predicted MWs of oleosin, MP, GFP and 4×H is-cassette (with 14 His-codons in each cassette) are 18.5, 30, 30, and 2.5 kDa, respectively. The appropriate MWs of fusion proteins can be calculated from these values as the sum of the MWs of each of the fusion components combined. These fusion proteins were shown to be identical in size to their predicted molecular weight, when examined with SDS-PAGE gel electrophoresis and by Western blot analysis. Stability of the transgene-encoded protein products (oleosin-His and MP-His) was investigated by Western blot analysis. Analysis of (selfed) plant generations (3^{rd} generation) after transformation (Table 1) showed clearly that transgene expression was stable over successive generations. Moreover, Western blot analysis of those transformed plants that were examined further revealed that the size of protein product remained constant regardless of the plant generation. These analyses also showed that the amount of fusion protein product was relatively constant between plant generations, indicative of an acquired stable expression level relative to other plant proteins.

Transformed plants may be analysed using antibodies to the carrier protein, reporter proteins or amino acids, respectively, whichever is applicaple for obtaining the desired result. Some seeds may be directly analysed for the selected amino acid content. Generally, a sequence of events was followed in which the results of the previous set of experiments were analyzed and only those constructs that were functional and suitable for later work were selected to be used in the later steps. Thus prior to the final plant transformation for production purposes, many aspects were analysed and checked by per se conventional methods to obtain the desired result. The constructs with the desired properties, including stable transformation of plants expressing stable amino acid-enriched protein are obtainable by the method describe herein,

The reporter protein enables easy and accurate selection in a cell-free in vitro translation (IVT) system of constructs that provide reliable expression of stable amino acid-enriched carrier proteins having a detectable reporter protein combined with a transient expression system. Said transient expression system enables verification that the biological function(s) of the carrier protein are not compromised. In the case of oleosin, the intact biological function means that the reporter protein is expressed in oil bodies of seeds, particularly in the membranes and cell walls of seed cells.

Those constructs exhibiting normal biological function(s) of the carrier protein as visualized by the reporter protein were selected and the nucleotide sequence encoding the reporter protein was removed. Accordingly, the nucleotide sequence encoding the carrier protein and the codons encoding the desired amino acids are fused in-frame.

The satisfactory constructs were selected and transferred into Agrobacterium, and positive (construct-containing) clones were selected by appropriate conventional means, e.g. by dot blot analysis, Southern blot hybridization and used for transformation of plants, preferably crop plants, particularly crop plants of the family Cruciferae. The transformed crop plants expressed stable amino acid-enriched proteins and accumulated the said proteins in the targeted or selected plant tissue(s).

The regulatory sequences, including the transcriptional promoter, were selected among regulatory sequences including tissue- or organ-specific transcription promoters, which enable targeting of mRNA synthesis to leaf, seed, or other desired or selected plant organs and, thereby, provide targeted accumulation of amino acid-enriched protein(s) in said organs. In order to enable the selection of satisfactory constructs, the amino acid codon-enriched sequence coding for selected, desired amino acid residues is placed in-frame between the carrier protein gene and the nucleotide sequence encoding a detectable reporter gene, preferably a fluorescent reporter protein, e.g. green fluorescent protein (GFP), β-glucuronidase or luciferase.

The preferred crop plants of the present invention belong to the family Brassicaceae, wherein amino acid-enriched, particularly histidine-enriched proteins, based on their ability to be anchored to and localized in the membranes of oil-bodies after expression, provide targeted accumulation. Based on the same principles, other constructs enabling accumulation of any other amino acid-enriched proteins in any other desired plant species and their tissues can be obtained.

The above objectives are accomplished by transforming plants with constructs comprising nucleotide sequences encoding carrier proteins selected from relatively small plant proteins functioning in selected secretory intracellular trafficking pathways enabling accumulation in selected, targeted plant organs, e.g. leaf membranes, seed membranes, cell walls of leaves or seed cells. This is exemplified in the present invention by two distinct carrier proteins, the oleosin protein and the TMV MP protein comprising a polyamino acid tail fused in-frame to said native carrier protein subsequently accumulating in selected targeted tissues of said transgenic plants. Particularly preferred nucleotide sequences coding for the carrier proteins are oleosins capable of accumulating in leaves or seed membranes and movement proteins, such as TMV MP, which can accumulate in cell walls of leaves or seed cells, when transcription is driven by an appropriate promoter. The nucleotide sequences encoding the carrier proteins are preferably obtained from plant species, which are not endogenous to the transformed plant host.

The nucleotide sequence encoding one or more amino acids is conveniently provided as one or more cassettes, which are carrying the selected amino acid codons, which encode the selected peptide entailing the carrier protein. The cassette, comprising nucleotide sequences encoding amino acid residues is preferably located such that it does not disturb the normal biological function(s) of the carrier protein, i.e. the 3′-terminal end of the carrier protein. If the amino acid codons are placed at the N-terminal end or somewhere in the middle of the carrier protein, the amino acid-enriched protein may not accumulate in the targeted organ as desired. The nucleotide sequence encoding the reporter protein is fused in the same translational frame with the extended nucleotide sequence enriched with the selected amino acid codons but lacking the termination codon. The optimal number of amino acid codons is about ten to eighty and is determined by checking correct protein translation in a cell-free translation system.

Therefore, in a preferred exemplified embodiment of the invention the nucleotide sequence encoding the carrier protein is positioned in the 5′-proximal end of the nucleotide sequence and the reporter nucleotide sequence is positioned in the 3′-terminal end. The preferred carrier proteins or nucleotide sequence of interest in the present invention are OLE or TMV 30K MP.

The OLE used in the examples of the present invention is a nucleotide sequence corresponding to the chromosomal gene of Arabidopsis thaliana coding for the seed protein oleosin (Ole), which is a component of membranes of oil-bodies in members of Cruciferae family. TMV 30K MP is a nucleotide sequence derived from the genomic RNA of Tobacco mosaic virus U1 (TMV U1). The gene encodes a non-structural hydrophobic 30K protein responsible for the movement of the viral genome from cell to cell in an infected plant (movement protein, MP) through plasmodesmata (PD). The 30K MP is targeted to and accumulates in cell walls and also in PD, and is expressed in plants transgenic for the MP gene.

The optimal number of amino acid codons is determined by randomly transforming constructs with nucleotide sequence encoding the carrier protein with extensions of different sizes, comprising different numbers of amino acid codons, into plants and selecting transgenic plants expressing proteins with undisturbed biological functions as compared to the native unmodified protein, assessed using a transient expression assay. Functional constructs and successful transformation can easily be demonstrated by the expression and intracellular localization of the expressed proteins, particularly the reporter protein, in transgenic plants.

In the present invention genetic engineering methods are used, which methods permit rapid identification and selection of chimeric constructs having an optimal codon content allowing stable accumulation of the amino acid enriched protein in a target plant tissue.

In the present invention the method for producing amino acid-enriched proteins in transgenic plants involves the preparation of one or more of the constructs described above.

The preferred selection method comprises expression of a fluorescent recombinant amino acid-enriched carrier protein from constructs such as (Ole-polyamino acid-GFP or TMV MP-poly-amino acid-GFP) in a cell-free translation system. The cell-free translation system used in this invention is a method that unequivocally identifies correct constructs and expression of the recombinant amino acid-enriched proteins. In addition to conventional detection methods, the detection of fluorescence, provided by GFP, or other reporter proteins, can be carried out using confocal laser scanning microscopy of seeds and/or leaves. The use of confocal laser scanning microscopy provides a convenient tool for early detection and selection of transgenic plants expressing amino acid-enriched proteins in-frame with the reporter protein.

The reason for using a reporter nucleotide sequence insert was that this protein could be used as a marker to confirm that the constructs and the expression of the amino acid-enriched carrier protein, e.g. oleosin-polyamino acid-GFP or TMV MP-poly-amino acid-GFP, from recombinant sequences were correct. Fluorescence, particularly GFP-fluorescence can be monitored by confocal laser scanning microscopy that allows detection of Ole-polyamino acid and MP-polyamino acid expression in vivo as well as determination of their expression levels.

Plant expression vectors comprise nucleotide sequences encoding carrier proteins, such as Ole and TMV MP fused to polyamino acid-coding sequences of varying length and carrying a reporter gene, e.g. a GFP gene positioned in the 3′-terminal end. For the construction of the plant expression vectors, said constructs were fused with sequences of varying lengths enriched with desired amino acid codons, e.g. for histidine the codons CAC and CAU, for cysteine and methionine the codons TGT, TGC and ATG, for glycine the codons GGA, GGT, GGC, and GGG, and for lysine the codons are AAA and AAG. These constructs lacked a termination codon. Stable constructs can be selected by providing constructs with a randomly selected number of amino acid codons, e.g. at least two amino acid codons, inserted as at least one cassette, but preferably more, for example two, four, six or eight cassettes and analyze the results obtained. If the cassette, for example, comprises 14 histidine codons (14×), this results in amino acid-entriched carrier proteins having 28(2×), 56(4×) and 112 (8×) His-codons downstream of the gene encoding the carrier protein (Ole or MP). Preliminary tests indicated that 8× (112 amino acids) were unstable producing deletion variants (mini-plasmids). Also some of the 6× (84 aa) clones were unstable. Thus, for further work clones 2×, 4× and 6× were fused with the 3′-proximal GFP gene with 3′-termination sequences. In precisely the same way it is possible to determine the optimal number of any amino acid codons, by inserting varying numbers of amino acid codons into the construct comprising the nucleotide sequence encosing the carrier protein and select constructs providing stable results.

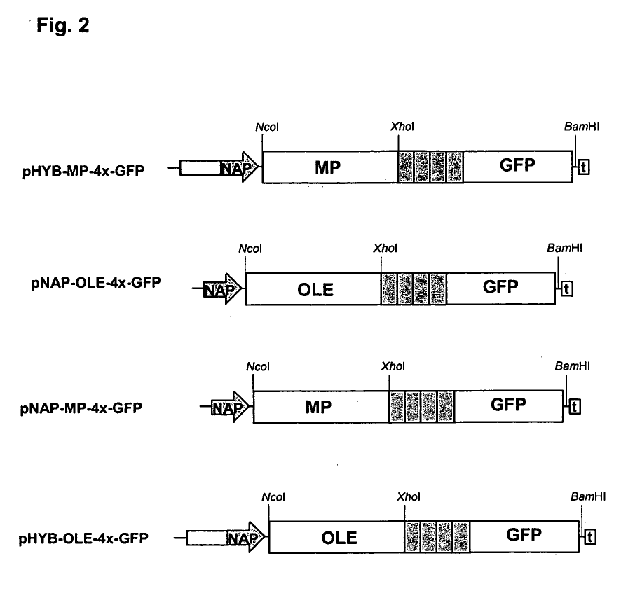

Constructs, such as those shown in FIG. 1, comprising regulatory sequences, particularly promoters such as the napin promoter NAP, the 35S CaMV promoter or a hybrid HYB promoter were operably linked to a carrier protein, particularly Ole and/or TMV MP. All of the constructs comprised a nucleotide sequence encoding a reporter protein, particularly preferred is the green fluorescent protein (GFP), as a marker for selection. The GFP gene was fused in-frame to the amino acid-tail, which was of varying length including one or more cassettes comprising at least two amino acid codons encoding the desired amino acids. Particularly, multiples of the cassettes were used (2×, 4×, 6×). Fluorescence of the reporter protein in transient expression assays was used in preliminary experiments to identify and select the best functional constructs. The transient expression assays included micro-projectile bombardment of crop plant embryos as exemplified by experiments involving particle bombardment of B. campestris embryos and Nicotiana benthamiana leaves, and shown in Table 1. The assays demonstrated that the NAP promoter was active in embryos, whereas the HYB promoter was efficient in both embryos and epidermal leaf cells. These data formed the basis for selection of four (of the said available eighteen constructs, FIG. 1) for subsequent work with transformations (FIG. 2).

The preferred transformation system was Agrobacterium-mediated transformation. Each gene construct described above was transferred into Agrobacterium tumefaciens; transformed clones were selected by Southern blot hybridization. Each of the transformed strains of Agrobacterium was then used to transform oilseed spring rape (B. campestris). Subsequent selection of transgenic plants expressing the amino acid-enriched carrier proteins (Ole-polyamino acid or MP-polyamino acid) was performed by:

(a) PCR, with primers specific for the selected amino acid codon-containing Ole and TMV MP genes

(b) Western blotting with antibodies raised against oleosin and 30K TMV MP

(c) Detection of GFP fluorescence in leaves, flowers, embryos.

Using the above experimental approach, when clones expressing proteins with undisturbed function have been found, the reporter nucleotide sequences can be removed from the corresponding construct(s) and the actual crop plants transformed with the said functionally proven, construct(s).

The aim of the invention is to provide a composition comprising an amino acid enriched carrier protein entailed with stable amino acid cassettes in plant material, particularly anchored to the cell walls and in the oilcake obtained subsequent to recovering the oil from oil plants. In embodiments of our invention, a stable amino acid-enrichment in seed proteins could be demonstrated.

The principles of the invention can be carried out by a multitude of known recombinant DNA techniques, which are available today. Therefore the following summary of the invention demonstrates a preferred embodiment.

The invention is described in more detail in the following examples. The production of amino acid-enriched protein is exemplified by histidine, methionine, cysteine, glycine and lysine, but the principles disclosed in the invention can be applied to any other desired amino acid(s) by inserting cassettes comprising a selected number of desired amino acid codons for any specified purposes.

### EXAMPLE 1

### Chromosomal DNA Isolation and PCR

Chromosomal DNA was isolated as follows. 300 μg of A. thaliana leaf tissue was homogenized in a mortar in liquid nitrogen to a fine powder. Then 3 ml of 100 mM Tris-HCI; pH 8.0, 500 mM NaCl, 50 mM EDTA was added, with further grinding after the addition of 600 μl of 10% SDS and 500 μl of 20% polyvinylpyrrolidone (average mwt 360,000). The mixture was transferred to a polypropylene tube and incubated at 65° C. for 10 min. Then 450 μl of ice-cold potassium acetate was added to the solution and the mixture was gently shaken by tube inversion, incubated on ice for 30 min and centrifuged for 10 min at 8000 rpm, 4° C. The supernatant was extracted with phenol/chloroform (1:1), and the DNA was precipitated using iso-propanol. The precipitated DNA was washed with 70% ethanol, and air-dried prior to being dissolved in 10 mM Tris-HCl; pH 8.0. The PCR reaction mixture was composed of 1×PCR buffer (10 × buffer: 500 mM KCl, 100 mM Tris-HCl; pH 9.0 at 25° C. and Triton X -100 ) with 1.5 mM MgCl₂, 1 μl of Taq polymerase (5U/fil), 0.2 mM of each dNTPs, primers at 0.4 μM and 3 μg of A. thaliana genomic DNA in 25 μl reaction volume. The template was denatured with heating for 3 min at 95° C., and 30 cycles of PCR were carried out with iCycler™ (Bio-Rad) thermal cycler with denaturation at 95° C. for 1 min, primer annealing at 65° C. for 1.5 min, primer extension at 72° C. for 2 min, with a final elongation step after 30 cycles at 72° C. for 10 min. The PCR product of expected size (776 bp) was isolated from a 1% agarose gel after electrophoresis of the final PCR reaction mix, purified using a gel slice kit (Qiagen), and then treated with 1 unit of T4 DNA polymerase in 1×T4 DNA polymerase reaction buffer (50 mM NaCl, 10 mM Tris-HCl; pH 7.9, 10 mM MgCl₂, 1 mM dithiothreitol) supplemented with 100 mM dNTP at 14° C. for 15 minutes prior to cloning.

### EXAMPLE 2

### Synthesis and Cloning of a Histidine Codon-enriched DNA Sequence

To construct a histidine codon-enriched DNA fragment, three chemically synthesized ssDNA segments were used:

H-P-1 (SEQ ID NO:1)
5′-GCGCCTCGAGTTCACCATCACCATCACCATCACGGGCACCATCAC,
H-P-2 (SEQ ID NO:2)
5′-CATCACCATCACCATGG
and
H-M (SEQ ID NO:3)
5′-CCGGATCCTAAAGTCGACCATGGTGATGGTGATGGTGATGGTGCC.

To obtain the dsDNA fragment carrying the His codon-enriched sequence, oligonucleotides H-M and H-P-2 were allowed to anneal in 1×AMV reverse transcriptase (RT) reaction buffer (50 mM Tris-HCl; pH 8.3, 50 mM KCl, 10 mM MgCl₂, 0.5 mM spermidine, 10 mM DTT) for 30 min at room temperature, and chain elongation reaction was carried out in the presence of 1×AMV RT buffer, 1 mM dNTPs and 1 unit of AMV RT for 45 min at 37° C. The reaction product was separated from non-annealed oligonucleotides by agarose gel electrophoresis, purified, and annealed with the oligonucleotide H-P-1 in 1×DNA polymerase I large (Klenow) fragment buffer (50 mM Tris-HCl; pH 7.2, 10 mM MgSO₄, 0.1 mM D TT) for 30 min at room temperature. The chain elongation reaction was performed with 5 units of Klenow polymerase in the presence of 25 mM of each dNTPs in 1×Klenow buffer for 30 min at 37° C. The dsDNA fragment of expected size (79 bp) was excised from a agarose gel (after appropriate electrophoretic separation), purified digested with Xhol and BamHI, and cloned into the similarly digested cloning vector pGEM-7Zf(+) (Promega Corporation, USA; catalogue number P2251). After restriction analysis and sequencing, clone pGEM-His-24 was selected for further manipulations. This clone contained a DNA segment that could potentially code for a 19 amino acid-long peptide containing 14 His residues (FIG. 3a). The sequence of this DNA fragment was flanked by Xhol and BamHI sites and contained also a SalI restriction site designed for subsequent cloning steps (FIG. 3a).

### EXAMPLE 3

### Synthesis and Cloning of a Cysteine and Methionine Codon-enriched DNA Sequence

To construct a cysteine and methionine codon-enriched DNA fragment, two chemically synthesized ssDNA segments were used:

C-M-P-1 (SEQ ID NO:4)
5′ CACCTCGAGTATGTTGTTGCATGTGCATGTGCTGTTGCATGTCGACAAAC 3′
and
C-M-P-2 (SEQ ID NO:5)
5′ GTTTGTCGACATGCAACAGCACATGCACATGCAACAACATACTCGAGGTG 3′

To obtain the dsDNA fragment carrying the Cys/Met codon-enriched sequence, oligonucleotides C-M-P-1 and C-M-P-2 were allowed to anneal in 1×DNA polymerase I large (Klenow) fragment buffer (50 mM Tris-HCl; pH 7.2, 10 mM MgSO₄, 0.1 mM DTT) for 30 min at room temperature. The dsDNA fragment of expected size (50 bp) was excised from a agarose gel (after appropriate electrophoretic separation), purified, digested with XhoI and BamHI, and cloned into the similarly digested cloning vector pGEM-7Zf(+) (Promega Corporation, USA; catalogue number P2251). After restriction analysis and sequencing, clone pGEM-Cys/Met-10 was selected for further manipulations. This clone contained a DNA segment that could potentially code for 16 amino acid-long peptide containing 10 Cys/Met-amino acid residues (FIG. 3b). The sequence of this DNA fragment was flanked by XhoI and BamHI sites and contained also a SalI restriction site designed for subsequent cloning step (FIG. 3b).

### EXAMPLE 4

### Synthesis and Cloning of a Glycine Codon-enriched DNA Sequence

To construct a glycine codon-enriched DNA fragment, two chemically synthesized ssDNA segments were used:

GL-P-1 (SEQ ID NO:6)
5′-GCGCCTCGAGTTGGTGGAGGTGGAGGCGGTGGAGGTGGCGTCGACAA
ATGGATCCCC-3′
and
GL-P-2 (SEQ ID NO:7)
5′-GGGGATCCATTTGTAGACGCCACCTCCTCCACCGCCTCCACCTCCAC
CAACTCGAGGCGC-3′

To obtain the dsDNA fragment carrying the glycine codon-enriched sequence, oligonucleotides GL-P-1 and GL-P-2 were allowed to anneal in 1×DNA polymerase I large (Klenow) fragment buffer (50 mM Tris-HCl; pH 7.2, 10 mM MgSO₄, 0.1 mM DTT) for 30 min at room temperature. The dsDNA fragment of expected size (60 bp) was excised from agarose gel (after appropriate electrophoretic separation), purified, digested with XhoI and BamHI, and cloned into the similarly digested cloning vector pGEM-7Zf(+) (Promega Corporation, USA; catalogue number P2251). After restriction analysis and sequencing, clone pGEM-Gly-9 was selected for further manipulations. This clone contained a DNA segment that could potentially code for a 19 amino acid-long peptide containing 9 Gly-amino acid residues (FIG. 3c). The sequence of this DNA fragment was flanked by XhoI and BamHI sites and contained also a SalI restriction site designed for subsequent cloning steps (FIG. 3c).

### EXAMPLE 5

### Synthesis and Cloning of a Lysine Codon-enriched DNA Sequence

To construct a lysine codon-enriched DNA fragment, two chemically synthesized ssDNA segments were used:

L-P-1 (SEQ ID NO:8)
5′-GCGCCTCGAGTTAAAAAGAAAAAGAAAAAGAAAAAGAAAAAGAAAAA
GGTCGACAAATGGATCCCC-3′
and
L-P-2 (SEQ ID NO:9)
5′-GGGGATCCATTTGTCGACCTTTTTCTTTTTCTTTTTCTTTTTCTTTT
TCTTTTTAACTCGAGGCGC-3′

To obtain the dsDNA fragment carrying the lysine codon-enriched sequence, oligonucleotides L-P-1 and L-P-2 were allowed to anneal in 1×DNA polymerase I large (Klenow) fragment buffer (50 mM Tris-HCl; pH 7.2, 10 mM MgSO₄, 0.1 mM DTT) for 30 min at room temperature. The dsDNA fragment of expected size (66 bp) was excised from agarose gel (after appropriate electrophoretic separation), purified, digested with XhoI and BamHI, and cloned into the similarly digested cloning vector pGEM-7Zf(+) (Promega Corporation, USA; catalogue number P2251). After restriction analysis and sequencing, clone pGEM-Lys-12 was selected for further manipulations. This clone contained a DNA segment that could potentially code for a 22 amino acid-long peptide containing 12 lysine amino acid residues (FIG. 3d) The sequence of this DNA fragment was flanked by XhoI and BamHI sites and contained also a SalI restriction site designed for subsequent cloning (FIG. 3d).

### EXAMPLE 6

### Isolation of the Oleosin (ole) Gene from the Arabidopsis thaliana Chromosomal DNA using PCR and Subsequent Cloning

To clone the oleosin gene from Arabidopsis thaliana chromosomal DNA, two oligonucleotide primeres were chemically synthesized:

| | | |
|---|---|---|
| OLE-P | (SEQ ID NO:10) | 5′-AAAACCATGGCGGATACAGCTAGAGGAACCCATC |
| and | | |
| OLE-M | (SEQ ID NO:11) | 5′-GGGGCCATGGGAGTAGTGTGCTGGCCACCACGAGTAC. |

OLE-P and OLE-M were used as the primers for PCR of genomic DNA from Arabidopsis thaliana (Example 1). The resulting PCR fragment was blunt-ended, and cloned into SmaI site of pGEM-3Zf(+) (Promega Corporation, U.S.A; catalogue number P2271).

The recombinant clones were verified by restriction analysis and sequencing. For subsequent work, clone pOLE4H was selected. When compared to the published oleosin sequence (X62353), the sequence of clone pOLE4H contains one nucleotide substitution (with no changes in the encoded amino acid sequence) in the gene intron i.e. a G to A substitution at position 513 in the pOLE4H sequence compared with the published sequence (X62353).

OLE-3′XhoI (SEQ ID NO:12) 5′- GCGCCTCGAGAAGTAGTGTGCTGGCCACCAC. was chemically synthesized, and used as a PCR primer to amplify the oleosin coding region from plasmid pOLE4H; the PCR product was cloned into the XhoI site of pGEM7Zf(+) (Promega Corporation, USA; catalogue number P2251). After the cloning of the pOLE4H sequence using this primer, the sequence of the resulting clones was verified by sequencing to ensure that all oleosin gene coding properties were retained. Based on this analysis, clone pOLE4/11 was selected. pOLE4/11 contained a wild-type oleosin gene lacking a termination codon, flanked by NcoI and XhoI restriction sites suitable for subsequent cloning steps.

### EXAMPLE 7

### Isolation of the 30K Movement Protein Gene from Tobacco Mosaic Virus (TMV 30K MP) Genomic RNA using RT-PCR and Subsequent Cloning

To clone the 30K gene from TMV genomic RNA, two specific oligonucleotide primers

Oligonucleotide primer N30K (SEQ ID NO:13)
5′-GCGGAATTCCCATGGCTCTAGTTGTTAAAGG)
and
Oligonucleotide primer C30K (SEQ ID NO:14)
5′-AGACCTCGAGGAAACGAATCCGATTCGGCGAC

were chemically synthesized; these primers contained an EcoRI restriction site and XhoI restriction site, respectively. The first strand cDNA was synthesized from TMV genomic RNA using 20 nM of C30K primer, 1 mM dNTPs and 1 unit of AMV in 1×AMV reverse transcriptase (RT) reaction buffer (50 mM Tris-HCl; 8.3, 50 mM KCl, 10 mM MgCl₂, 0.5 mM spermidine, 10 mM DTT) for 45 min at 37° C. After that 5 μl of reaction mix was subjected to PCR reaction in 1×PCR buffer (10 × buffer: 500 mM KCl, 100 mM Tris-HCl; pH 9.0 at 25° C. and Triton ×-100 ) with 1.5 mM MgCl₂, 2 μl of Taq polymerase (5U/μl), 0.2 mM of each dNTPs, primers at 0.4 μM in 25μl reaction volume. The template was denatured with heating for 3 min at 95° C., and 28 cycles of PCR were carried out with iCycler (Bio-Rad) thermal cycler with denaturation at 95° C. for 1 min, primer annealing at 68° C. for 1.5 min, primer extension at 72° C. for 2 min, with a final elongation step after 30 cycles at 72° C. for 10 min. The resulting DNA product was cleaved by EcoRI and XhoI and cloned into EcoRI-XhoI-digested pGEM-7Zf(+) (Promega Corporation, USA; catalogue number P2251). Following restriction analysis of the recombinant clones and sequencing, plasmid pGEM-30K was selected for further work. In this plasmid, the termination codon of the TMV 30K MP gene was replaced by a XhoI site for subsequent fusion with the His, Cys/Met, Gly and Lys codon-enriched sequence (Examples 2-5; FIG. 3a-d).

### EXAMPLE 8

### Microprojectile Bombardment

Microprojectile (particle) bombardment was performed using the rupture disk method with a high-pressure helium-based apparatus PDS-1000 (Bio-Rad) as described in Morozov et al. (1997). Tungsten particles were prepared by vortexing of 60 mg in 70% ethanol for 3-5 minutes, followed by incubation on a bench for 15 min. Mixture was pelleted by short spinning and the supernatant was removed. Sterile water was added onto pellet and vortexed for 1 min. Particles were allowed to settle for 1 minute and spinned for 2 s. The supernatant was removed. This was repeated three times. Sterile 50% glycerol was added to bring particle concentration to 60 mg/ml. For each particle bombardment, DNA was precipitated onto tungsten particles (M-20, 1.3μ) with calcium chloride and ethanol. 5-10 μg of plasmid DNA, 50 μl of CaCl₂ (2.5 M) and 20 μl of spermidine (0.1 M) were mixed and vortexed for 2-3 min, allowed to settle and spinned for 2 s. Supernatant was removed and 140 μl of 70% ethanol was added onto surface of pellet, removed and 100% ethanol added and removed without disturbing the pellet, and repeated. 6 μl of suspension was pipetted onto macrocarrier and used for bombardment. A detached leaf of Nicotiana benthamiana (15-30 mm size) was placed in the center of a plastic Petri dish and bombarded on a solid support at a target distance of 7 cm. Bombardment was done with a pulse of 1350 kPa helium gas in a vacuum chamber. Inoculated leaves were analyzed 24 hours after particle bombardment. GFP fluorescence was monitored using a confocal laser scanning imaging system MRC-1024 (Bio-Rad).

### EXAMPLE 9

### Construction of an E. Coli Strain Over-expressing the C-terminal Hydrophilic Terminus of Oleosin for Subsequent Rabbit Immunization and Obtaining Oleosin-specific Antibodies

Most of the oleosin N-terminal sequence region is hydrophobic, whereas the amino acid sequence of the oleosin C-terminal portion encoded by the second exon of the oleosin gene represents hydrophilic sequence that is suitable for expression in E. coli and using as the antigen in immunizations. Therefore, the C-terninal oleosin region (aa 120-173) was selected for this purpose. The second exon of the oleosin gene was amplified in a PCR reaction using the plasmid pOLE4/11 (Example 6) as the template and the oligonucleotide primers:

| | | |
|---|---|---|
| OLE-EX-B | (SEQ ID NO:15) | (5′-TGTGGGATCCTACGCAACGGGAGAGCACCCA) |
| and | | |
| OLE-3′XhoI | (SEQ ID NO:12) | (5′-GCGCCTCGAGAAGTAGTGTGCTGGCCACCAC) |

containing BamHI restriction site and XhoI restriction site, respectively. The resulting PCR product was digested with BamHI and XhoI and cloned into XhoI- and BamHI-digested pGEM-7Zf(+) (Promega Corporation, USA; catalogue number P2251). After restriction analysis of the resulting clones and sequencing, clone pGEM-OLE2EX was selected for the subsequent cloning steps. To obtain an expression construct, the region of the oleosin second exon was removed from pGEM-OLE2EX by digestion with BamHI and XhoI and cloned into the (BamHI and SalI digested) expression vector pQE30 (QIAGEN). In the resulting construct pQE-OLE2EX, the 3′-terminal portion of the oleosin gene was translationally fused to a leader sequence containing a 6×His tag, which makes it possible to affinity purify the expressed product. The plasmid pQE-OLE2EX was transformed into E. coli strain M15 [pREP4] (QIAGEN). Induction of recombinant protein expression was carried out in Luria broth by adding IPTG to a final concentration 1 mM followed by incubation on a rotary shaker at 220 rpm for 2-4 h at 37° C. The E. coli cells were collected from induced and non-induced control cultures, and expression of the recombinant protein was analyzed by SDS-PAGE. For SDS-PAGE, 12.5% acrylamide gels were prepared. Gels were run at 100V/500 mA for 45 1 h, and subsequently stained in CBB (0.05% Coomassie Brilliant Blue, 50% methanol, 7% glacial acetic acid) and destained in 7% methanol/5% acetic acid. In the induced cultures, a major band was evident that was absent in the non-induced cultures. Mobility of this band corresponded to the expected mobility of the H is-tagged C-terminal portion of the oleosin gene (8.3 kDa).

### EXAMPLE 10

### In Vitro Translation System

A cell-free in vitro translation (IVT) system enabled the selection/identification of the optimal number of codons that could be translated correctly. The IVT system consists of a wheat germ extract containing all the components (e.g. 70S or 80S ribosomes, tRNAs, aminoacyl-tRNA synthetases, initiation, elongation and termination factors) required for translation of exogenous RNA even in the presence of low concentrations of double-stranded RNA (dsRNA) or oxidized thiols. The extract also contains requisite amino acids, energy sources (ATP, GTP), energy regenerating systems (creatine phosphate and creatine phosphokinase) and other essential co-factors (Mg²⁺, K⁺, etc.).

Clones with 2, 4 and 6 repeats of the His codon-enriched sequences were selected for IVT analysis. Plasmids pMP-1×, pMP-2×, pMP-4×, and pMP-6× contained the 30 K MP gene fused to His-coding sequences of different repeat lengths, placed under the control of a T7 promoter. Plasmids were linearized with BamHI, and used as template in IVT with T7 RNA polymerase. Equal amounts of the resulting transcripts were translated in a wheat germ cell-free system (TNT® T7/SP6 Coupled Wheat Germ Extract System L5030, Promega corporation). It was consistently observed that transcripts corresponding to clones with one, two and four repeats of the His-enriched sequence (MP-1×, MP-2×, and MP-4×) gave rise to products of predicted molecular mass, whereas transcripts potentially coding for the protein with 6 repeats of the His-enriched sequence (MP-6×) was translated inefficiently and gave rise to smeared products of low molecular mass. This approach for clone selection/identification is exemplified in the Examples 11-14.

### EXAMPLE 11

### Fusion of Histidine Codon-enriched DNA Sequences to the TMV 30K MP Gene and Testing the Correct Translation of His Codon-enriched Genes using an in Vitro Translation System

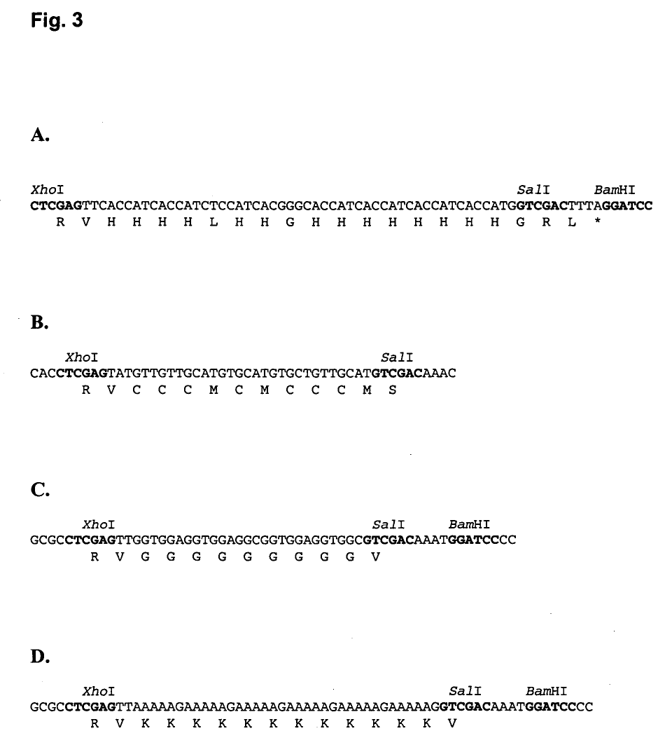

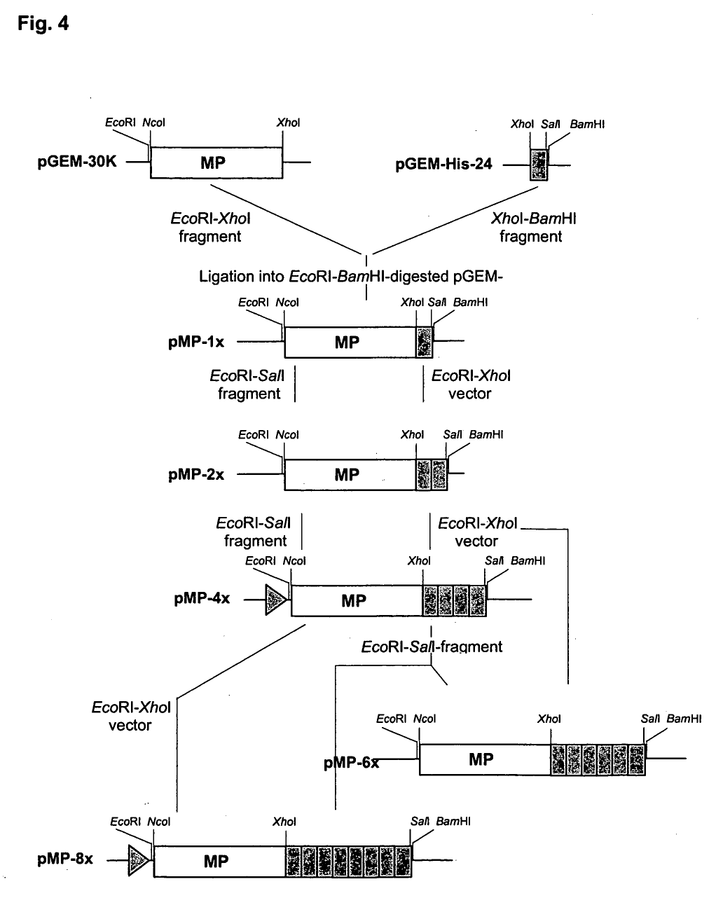

Fusions between His codon-enriched sequences and the TMV 30K MP gene were obtained by appropriate cloning of a combination of pGEM-His-24 (Example 2) containing the sequence coding for 14 His residues (=one cassette) (FIG. 3a), and plasmid pGEM-30K (Example 7) containing the TMV 30K MP gene lacking its natural terminator codon. To obtain His codon-enriched sequences of different lengths fused to the MP gene, a stepwise cloning procedure was carried out (FIG. 4). In the first step, the 30K MP gene was excised from pGEM-30K with EcoRI and XhoI, and the His-codon-enriched sequence from pGEM-His-24 was excised with XhoI and BamHI. Both DNA fragments were ligated into pGEM-3Zf(+) (Promega Corporation, USA; catalogue number P2271) digested with EcoRI and BamHI (FIG. 4). The resulting clone, designated pMP-1×, had one histidine-coding sequence unit (cassette) from pGEM-His-24 fused to the TMV 30K MP gene.

Additional stepwise cloning steps performed to increase the length of the His-coding “tail” in the MP fusion were based on the pMP-1× plasmid. These clonings used the restriction sites XhoI and SalI which have been designed in the His codon-enriched sequence of pGEM-His-24 to be situated to the reading frame of the encoded polypeptide. Together with the fact that digestions with XhoI and SalI produce identical sticky ends, this preserved the reading frame of the resulting addition-enlarged His-containing sequence in the further cloning steps described below. To obtain a plasmid with two histidine-coding sequence units from pGEM-His-24, pMP-2×, the EcoRI-SalI-fragrnent from pMP-1× was cloned into pMP-1× digested with EcoRI and XhoI, resulting in duplication of the histidine-coding sequence unit. Therefore, the resulting pMP-2× plasmid contained 24 histidine residues in the MP C-terminal “tail”. Subsequently, to obtain pMP4×, the EcoRI-SalI-fragment from pMP-2× was cloned into pMP-2× digested with EcoRI and XhoI; to obtain pMP-6×, the EcoRI-SalI-fragment from pMP-4× was cloned into pMP-2× digested with EcoRI and XhoI; and to obtain pMP-8×, the EcoRI-SalI-fragment from pMP-4× was cloned into pMP4× digested with EcoRI and XhoI. Specifically, similar clonings were repeated to obtain plasmids carrying 56, 84 and 112 histidine residues (4×, 6× and 8× cassettes) in the MP C-terminal “tail” (FIG. 4).

Clones with 112 His codons in the C-terminal “tail” (pMP-8x series) were unstable and gave rise to deletion variants (mini-plasmids) when grown in liquid medium. Lowered stability upon repeated growth in liquid medium was also observed for some of the clones with 84 histidines (pMP-6× series). Thus, for further experiments only clones with 2, 4 and 6 repeats of the His codon-enriched sequences were selected, pMP-2×, pMP4×, and pMP-6× (FIG. 4).

In plasmids pMP-1×, pMP-2×, pMP4×, and pMP-6×, the 30K MP gene fused to His-coding sequences of different lengths was placed under the control of the T7 promoter. These plasmids were linearized with BamHI, prior to in vitro transcription with T7 RNA polymerase. Equal amounts of the resulting transcripts were translated in a wheat germ cell-free system (TNT® T7/SP6 Coupled Wheat Germ Extract System L5030 Promega corporation, described in Example 10). It was found that transcripts of the clones with one, two and four repeats of the His-enriched sequence (trMP-1×, trMP-2×, and trMP4×) gave rise to the products of predicted, gradually increasing molecular masses, whereas the transcript potentially coding for a protein with 6 repeats of the His-enriched sequence (trMP-6×) was translated inefficiently and gave rise to smeared products of lower molecular mass. This experiment showed that translation of the His codon-enriched sequences of more than 56 residues by plant ribosomes occurs with errors, e.g. His codons could represent “hungry” codons where ribosomes stopped and jammed. On the basis of this experiment only clones with 2 and 4 repeats were selected for further work in vivo.

### EXAMPLE 12

### Fusion of Cysteine/methionine Codon-enriched DNA Sequences to the TMV 30K MP Gene and Testing the Correct Translation of Cys/Met Codon-enriched Genes using an in Vitro Translation System

Fusions between Cys/Met codon-enriched sequences and the TMV 30K MP gene were obtained by appropriate cloning of a combination of pGEM-Cys/Met-10 (Example 3) containing the sequence coding for 10 Cys/Met residues (=one cassette) (FIG. 3b), and plasmid pGEM-30K (Example 7) containing the TMV 30K MP gene lacking its natural terminator codon, as in example 10. To obtain Cys/Met codon-enriched sequences of different lengths fused to the MP gene, a stepwise cloning procedure was carried out (FIG. 4). In the first step, the 30K MP gene was excised from pGEM-30K with EcoRI and XhoI, and the Cys/Met-codon-enriched sequence from pGEM-Cys/Met-10 was excised with XhoI and BamHI. Both DNA fragments were ligated into pGEM-3Zf(+) (Promega Corporation, USA; catalogue number P2271) digested with EcoRI and BamHI (FIG. 4). The resulting clone, designated pMP-Cys/Met-1×, had one cysteine/methionine-coding sequence unit (cassette) from pGEM-Cys/Met-10 fused to the TMV 30K MP gene.

To obtain a plasmid with two cysteine/methionine-coding sequence units from pGEM-Cys/Met-10, pMP-Cys/Met-2×, the EcoRI-SalI-fragment from pMP-Cys/Met-1× was cloned into pMP-Cys/Met-1× digested with EcoRI and XhoI, resulting in duplication of the cysteine/methionine-coding sequence unit. Therefore, the resulting pMP-Cys/Met-2× plasmid contained 20 cysteine/methionine residues in the MP C-terminal “tail”. Subsequently, to obtain pMP-Cys/Met-4×, the EcoRI-SalI-fragment from pMP-Cys/Met-2× was cloned into pMP-Cys/Met-2× digested with EcoRI and XhoI; to obtain pMP-Cys/Met-6×, the EcoRI-SalI-fragment from pMP-Cys/Met-4× was cloned into pMP-Cys/Met-2× digested with EcoRI and XhoI; and to obtain pMP-Cys/Met-8×, the EcoRI-SalI-fragment from pMP-Cys/Met-4× was cloned into pMP-Cys/Met-4× digested with EcoRI and XhoI.

In plasmids pMP-Cys/Met-1×, pMP-Cys/Met-2×, pMP-CYs/Met4×, and pMP-Cys/Met-6×, the 30K MP gene fused to Cys/Met-coding sequences of different lengths was placed under the control of the T7 promoter. Plasmids were linearized with BamHI, and subjected to in vitro transcription with T7 RNA polymerase. Equal amounts of the resulting transcripts were translated in a wheat germ cell-free system (TNT® T7/SP6 Coupled Wheat Germ Extract System L5030 Promega corporation). It was found that transcripts of clones with one, two and four cassettes of the Cys/Met-enriched sequence (MP-Cys/Met-1×, MP-Cys/Met-2×, and MP-Cys/Met-4×) gave rise to the products of predicted, gradually increasing molecular masses, As exemplified in the example 11, the increase in the length of polyamino acid sequence led to a decreased stability of transcripts. For further experiments only clones with 2, 4 and 6 cassettes (MP-Cys/Met-2×, MP-Cys/Met4×and MP-Cys/Met-6×) of Cys/Met codon-enriched sequences were selected.

### EXAMPLE 13

### Fusion of Glycine Codon-enriched DNA Sequences to the TMV 30K MP Gene and Testing the Correct Translation of Gly Codon-enriched Genes using an In Vitro Translation System

Fusions between glycine codon-enriched sequences and the TMV 30K MP gene were obtained by appropriate cloning of a combination of pGEM-Gly-9 (Example 4) containing the sequence coding for 9 Gly residues (=one cassette) (FIG. 3c), and plasmid pGEM-30K containing the TMV 30K MP gene lacking its natural terminator codon, as in example 10. To obtain glycine codon-enriched sequences of different lengths fused to the MP gene, a stepwise cloning procedure was carried out as in Examples 11 and 12 and shown in FIG. 4.

The resulting first clone, designated pMP-Gly-1×, had one glycine-coding sequence unit (cassette) from pGEM-Gly-9 fused to the TMV 30K MP gene. Subsequently, this cassette was “multiplied” as in examples 11 and 12; to generate clones pMP-Gly-2×, pMP-Gly-4×, pMP-Gly-6×, and pMP-Gly-8×. To obtain a plasmid with two glycine-coding sequence units from pGEM-Gly-9, pMP-Gly-2×, the EcoRI-SalI-fragment from pMP-Gly-1× was cloned into pMP-Gly-1× digested with EcoRl and XhoI, resulting in duplication of the glycine-coding sequence unit. Subsequently, to obtain pMP-Gly-4×, the EcoRI-SalI-fragment from pMP-Gly-2× was cloned into pMP-Gly-2× digested with EcoRI and XhoI; to obtain pMP-Gly-6×, the EcoRI-SalI-fragment from pMP-Gly4× was cloned into pMP-Gly-2× digested with EcoRI and XhoI; and to obtain pMP-Gly-8×, the EcoRI-SalI-fragment from pMP-Gly-4× was cloned into pMP-Gly-4× digested with EcoRI and XhoI. Plasmids were linearized with BamHI, and subjected to in vitro transcription with T7 RNA polymerase and translated in a wheat germ cell-free system (TNT® T7/SP6 Coupled Wheat Germ Extract System L5030 Promega corporation, described in Example 10). As exemplified in example 11, the increase in the length of polyamino acid sequence led to a decreased stability of transcripts. For further experiments only the clones with 2 and 4 cassettes (MP-Gly-2×, and MP-Gly-4×) of glycine codon-enriched sequences were selected.

### EXAMPLE 14

### Fusion of Lysine Codon-enriched DNA Sequences to the TMV 30K MP Gene and Testing the Correct Translation of Lys Codon-enriched Genes using an In Vitro Translation System

Fusions between lysine codon-enriched sequences and the TMV 30K MP gene were obtained by appropriate cloning of a combination of pGEM-Lys-12 (Example 5)containing the sequence coding for 12 Lys residues (=one cassette) (FIG. 3d), and plasmid pGEM-30K containing the TMV 30K MU gene lacking its natural terminator codon, as in examples 11-13. To obtain lysine codon-enriched sequences of different lengths fused to the MP gene, a stepwise cloning procedure was carried out as in examples 11, 12 and 13 and shown in FIG. 4.

The resulting first clone, designated pMP-Lys-1×, had one lysine-coding sequence unit (cassette) from pGEM-Lys-12 fused to the TMV 30K MP gene. Subsequently, this cassette was “multiplied” as in examples 11, 12 and 13; to generate clones pMP-Lys-2×, pMP-Lys4×, pMP-Lys-6×, and pMP-Lys-8×, the EcoRI-SalI-fragment from pMP-Lys-4× was cloned into pMP-Lys-4× digested with EcoRI and XhoI. To obtain a plasmid with two lysine-coding sequence units from pGEM-Lys-12, pMP-Lys-2×, the EcoRI-SalI-fragment from pMP-Lys-1×, was cloned into pMP-Lys-1× digested with EcoRI and XhoI, resulting in duplication of the lysine-coding sequence unit. Subsequently, to obtain pMP-Lys-4×, the EcoRI-SalI-fragment from pMP-Lys-2× was cloned into pMP-Lys-2× digested with EcoRl and XhoI; to obtain pMP-Lys-6×, the EcoRI-SalI-fragment from pMP-Lys-4× was cloned into pMP-Lys-2× digested with EcoRI and XhoI; and to obtain pMP-Lys-8×, the EcoRI-SalI-fragment from pMP-Lys-4× was cloned into pMP-Lys-4× digested with EcoRl and XhoI. Plasmids were linearized with BamHI, and subjected to in vitro transcription with T7 RNA polymerase and translated in a wheat germ cell-free system (TNT® T7/SP6 Coupled Wheat Germ Extract System L5030 Promega corporation, described in Example 10). As exemplified in the Examples 10-12, the increase in the length of polyamino acid sequence led to a decreased stability of transcripts. For further experiments only the clones with 2 and 4 cassettes (MP-Lys-2×, and MP-Lys4×) of lysine codon-enriched sequences were selected.

### EXAMPLE 15

### Isolation of the Napin Promoter from the Arabidopsis thaliana Chromosomal DNA using PCR and Subsequent Cloning

To clone the napin promoter from the Arabidopsis thaliana chromosomal DNA, two oligonucleotide primers were chemically synthesized:

| | | |
|---|---|---|
| NAP-P | (SEQ ID NO:16) | 5′-TCTTACTCGAGTGAAACCAAATTAAC |
| and | | |
| NAP-M | (SEQ ID NO:17) | 5′-CTTGTTAGCCATGGTTTGCTATTTGTG. |

To facilitate subsequent cloning, the primer sequences contained XhoI and NcoI restriction sites. A. thaliana chromosomal DNA was isolated as described for isolation (Example 1) and cloning of the oleosin gene (Example 6). The PCR reaction was carried out as described above for the oleosin gene (Example 6) except that the concentration of MgCl₂ in the reaction mixture was 1.5 mM. The PCR product of expected size (369 bp) was isolated from a 1% agarose gel following electrophoresis. The PCR product was purified and treated with T4 DNA polymerase in the presence of dNTP at 14° C. as in example 1, and cloned into SmaI-digested pGEM-3Zf(+) (Promega Corporation, USA; catalogue number P2271). After sequencing, clones pGEM-NAP4 and pGEM-NAP9 were selected for subsequent work

### EXAMPLE 16

### Construction of Expression Plasmids Containing the Napin Promoter (NAP) or Chimeric Promoter (HYB), which Comprises the Entire Napin Promoter and Enhancer Region of the Cauliflower Mosaic Virus 35S Promoter

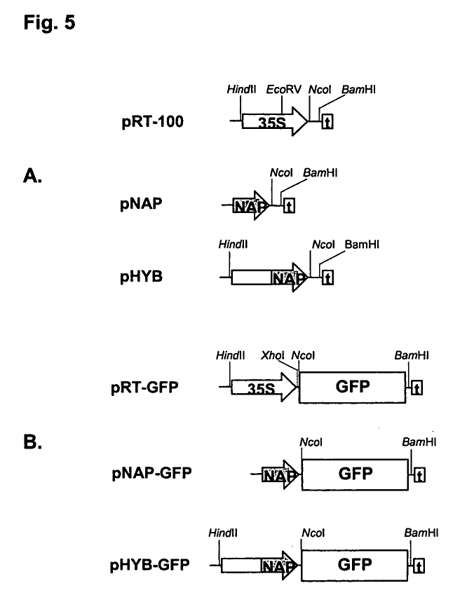

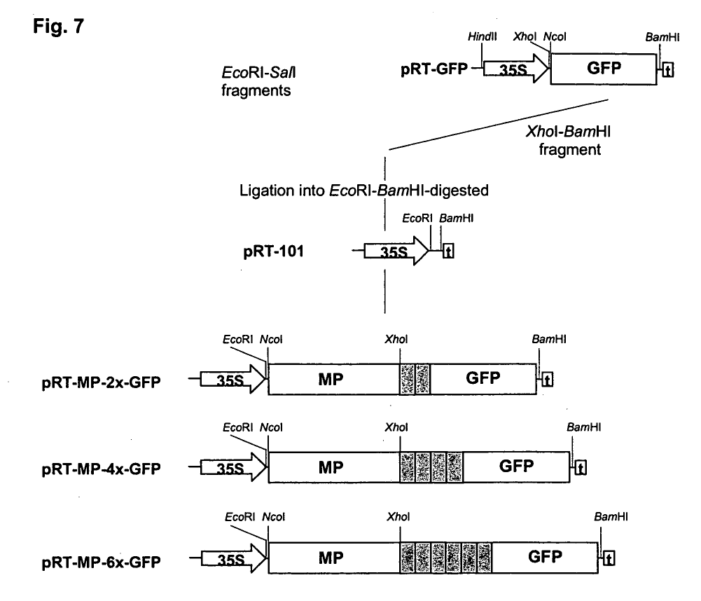

Plant expression vectors containing the napin promoter were constructed using plasmid pRT100 (Töpfer et al., Nucleic Acid Res. 15(14): 5890, 1987). A set of plant expression vectors for transcriptional and translational fusions. Nucleic Acids Res. 15(14): 5890, 1987). The basic strategy to make a fusion between MP and GFP with 2×-Gly (i.e. MP-Gly-2×-GFP) is shown in FIG. 7. This strategy utilizes XhoI/SalI restriction sites which have different cleavage site but similar 4-base protruding DNA ends. The GFP gene was excised from pRT-GFP as the XhoI-BamHI-fragment, and both DNA fragments were ligated into pRT101 (Töpfer et al., Nucleic Acids Res. 15(14): 5890, 1987). digested with EcoRI and BamHI to give the construct pRT-MP-2×-GFP. pRT100 contains the 35S promoter of Cauliflower mosaic virus (CaMV 35S promoter). For construction of pNAP, in which the whole sequence of the CaMV 35S promoter was removed and replaced by the sequence of the napin promoter, the napin promoter region was excised from the plasmid pGEM-NAP4 (Example 15) with Ecl136II and NcoI and ligated into pRT100 digested with HindII and NcoI (FIG. 5). To construct pHYB in which the sequence of the napin promoter replaced the promoter region of the 35S promoter retaining the 35S promoter enhancer region upstream of the inserted napin promoter sequence, pGEM-NAP9 (Example 15) was digested with XhoI, made blunt-ended with Klenow enzyme, and digested with NcoI. The resulting fragment was ligated into pRT100 digested with EcoRV and NcoI.

### EXAMPLE 17

### Cloning of His-enriched Oleosin and TMV 30K MP Genes into Expression Plasmids Containing the CaMV 35S, Napin, and Chimeric Promoters.

Detailed construction details are described in FIGS. 7 through to 12.

Initially, a set of constructs were obtained in which genes representing fusion proteins comprised of the TMV MP, His-enriched sequences, and GFP, were placed under the control of the 35S CaMV promoter (FIG. 7). His-enriched sequences of three different lengths (namely, those with two, four, and six repeats of the His-encoding sequence unit, Example 11) were selected. The MP gene fused to two His-coding units was excised from pMP-2× with EcoRI and SalI (FIG. 4), the GFP gene was excised from pRT-GFP as a XhoI-BamHI-fragment, and both DNA fragments were ligated into pRT101 digested with EcoRI and BamHI to give the construct pRT-MP-2×-GFP. Similarly, to obtain pRT-MP-4×-GFP, the MP gene fused to four His-coding units was excised from pMP4× with EcoRI and SalI (FIG. 4), the GFP gene was excised from pRT-GFP (See FIG. 7 for details) as a XhoI-BamHI-fragment, and both DNA fragments were ligated into pRT101 digested with EcoRI and BamHI. To obtain pRT-MP-6×-GFP, the MP gene fused to six His-coding units was excised from pMP-6× with EcoRI and SalI (FIG. 4), the GFP gene was excised from pRT-GFP as a XhoI-BamHI-fragment, and both DNA fragments were ligated into pRT101 digested with EcoRI and BamHI (FIG. 7).

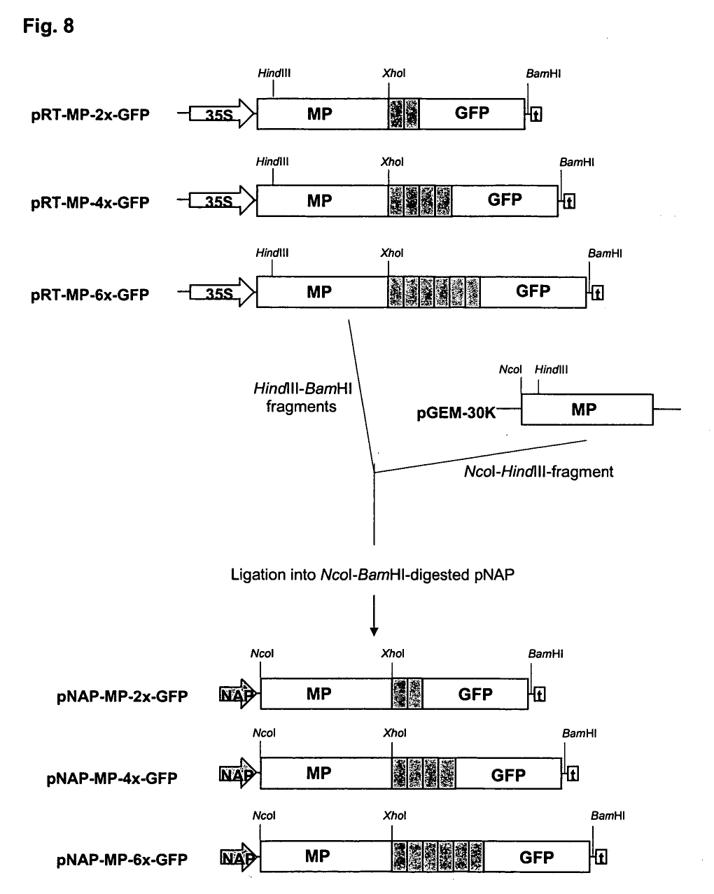

To obtain equivalent constructs under the control of the napin promoter (replacing the CaMV 35S promoter), the following cloning steps were carried out. The N-terminal portion of the TMV 30K MP gene was excised as a NcoI-HindIII-fragment from pGEM-30K; the fragment containing the rest of the M P gene fused to two H is-coding units and the GFP gene was excised from pRT-MP-2×-GFP with HindIII and XbaI, and both fragments were ligated into pNAP digested with NcoI and BamHI to give p NAP-MP-2×-GFP. In a similar fashion, to obtain pNAP-MP-4×-GFP, the N-terminal portion of the TMV 30K MP gene was excised as the NcoI-HindIII-fragment from pGEM-30K, and the fragment containing the rest of the MP gene fused to four His-coding units and the GFP gene was excised from pRT-MP4×-GFP with HindIII and XbaI, and both fragments were ligated into pNAP digested with NcoI and BamHI. Finally, to construct pNAP-MP-6×-GFP, the N-terminal portion of the TMV MP gene was excised as a NcoI-HindIII-fragment from pGEM-30K, and the fragment containing the rest of the MP gene fused to six His-coding units and the GFP gene was excised from pRT-MP-6×-GFP with HindIII and XbaI, and both fragments were ligated into pNAP digested with NcoI and BamHI (FIG. 8).

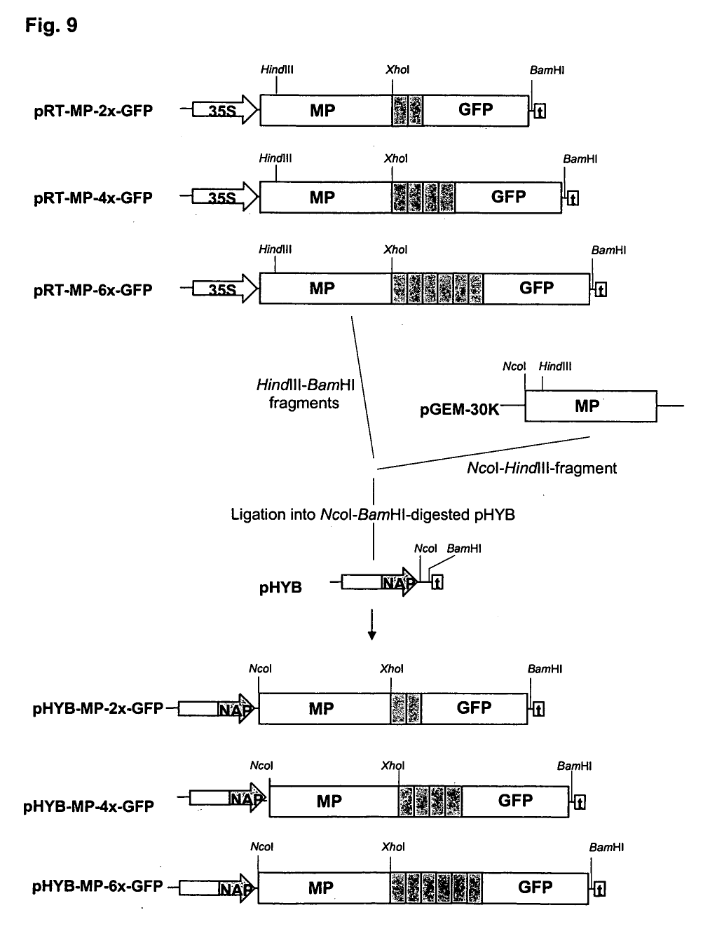

Similar cloning was undertaken to obtain constructs in which the MP-His-GFP fusion genes were placed under the control of the hybrid promoter. The N-terminal portion of the TMV MP gene was excised as a NcoI-HindIII-fragment from pGEM-30K, the fragment containing the rest of the MP gene fused to two His-coding units and the GFP gene was excised from pRT-MP-2×-GFP with HindIII and XbaI, and both fragments were ligated into pHYB digested with NcoI and BamHI to give pHYB-MP-2×-GFP. To obtain pHYB-MP-4×-GFP, the N-terminal portion of the TMV MP gene was excised as a NcoI-HindIII-fragment from pGEM-30K, the fragment containing the rest of the MP gene fused to four His-coding units and the GFP gene was excised from pRT-MP-4×-GFP with HindIII and XbaI, and both fragments were ligated into pHYB digested with NcoI and BamHI. To construct pHYB-MP-6×-GFP, the N-terminal portion of the TMV MP gene was excised as a NcoI-HindIII-fragment from pGEM-30K, the fragment containing the rest of the MP gene fused to six His-coding units and the GFP gene was excised from pRT-MP-6×-GFP with HindII and XbaI, and both fragments were ligated into pHYB digested with NcoI and BamHI. Based on the oleosin gene from pOLE4/11, three sets of expression vectors were constructed that had the CaMV 35S, napin, or hybrid promoters, each set containing His-coding regions of three different lengths (FIG. 9).

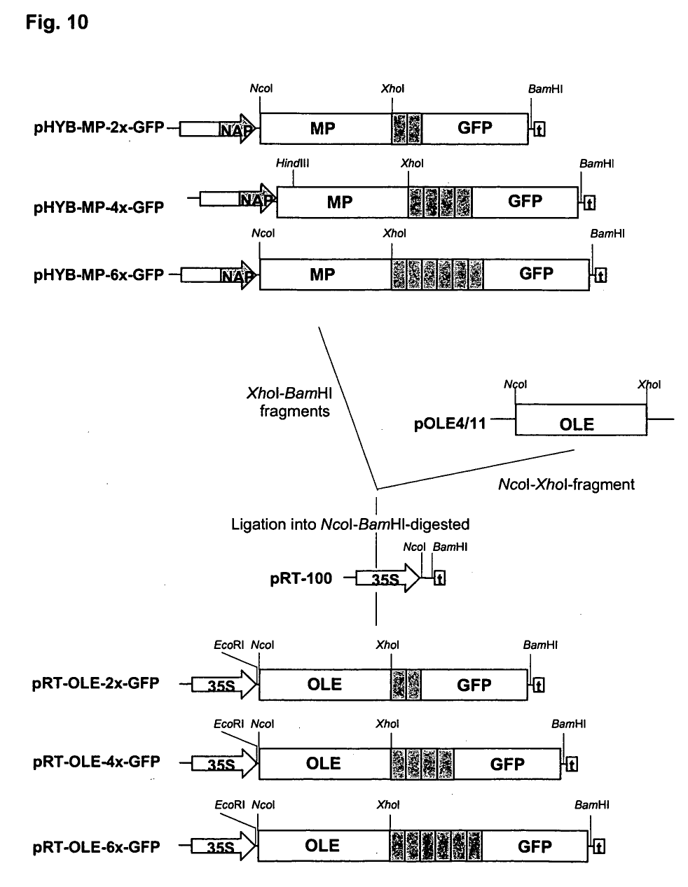

To construct expression vectors based on the CaMV 35S promoter, the following cloning procedures were carried out. The oleosin gene was excised from pOLE4/11 as a NcoI-XhoI-fragment, the region containing two His-coding sequence units fused to the GFP gene was excised from pHYB-MP-2×-GFP with XhoI and BamHI, and both DNA fragments were ligated into pRT100 digested with NcoI and BamHI resulting in the construct pRT-OLE-2×-GFP. To obtain pRT-OLE-4×-GFP, the oleosin gene was excised from pOLE4/11 as a NcoI-XhoI-fragment, the region containing four His-coding sequence units fused to the GFP gene was excised from pHYB-MP-4×-GFP with XhoI and BamHI, and both DNA fragments were ligated into pRT100 digested with NcoI and BamHI. To obtain pRT-OLE-6×-GFP, the oleosin gene was excised from pOLE4/11 as a NcoI-XhoI-fragment, the region containing six His-coding sequence units fused to the GFP gene was excised from pHYB-MP-6×-GFP with XhoI and BamHI, and then both DNA fragments were ligated into pRT100 digested with NcoI and BamHI (FIG. 10).

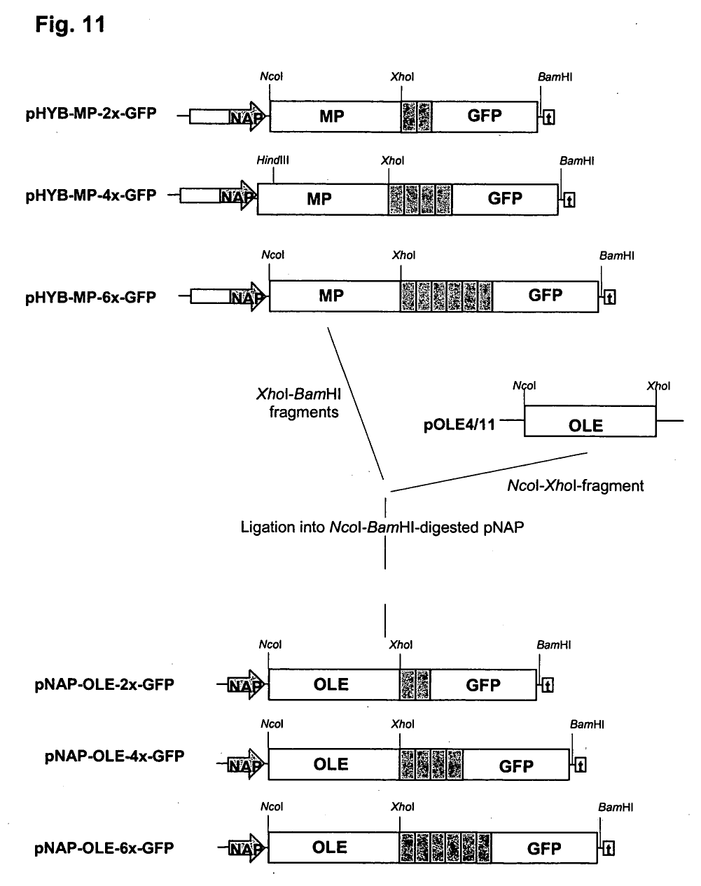

A similar cloning scheme was applied to construct expression vectors based on the napin promoter. The oleosin gene was excised from pOLE4/11 as a NcoI-XhoI-fragment, the region containing two His-coding sequence units fused to the GFP gene was excised from pHYB-MP-2×-GFP with XhoI and BamHI, and both DNA fragments were ligated into pNAP digested with NcoI and BamHI, resulting in construct pNAP-OLE-2×-GFP. To obtain pNAP-OLE-4×-GFP, the oleosin gene was excised from pOLE4/11 as a NcoI-XhoI-fragment, the region containing four His-coding sequence units fused to the GFP gene was excised from pHYB-MP-4×-GFP plasmid with XhoI and BamHI, and both DNA fragments were ligated into pNAP digested with NcoI and BamHI. To obtain pNAP-OLE-6×-GFP, the oleosin gene was excised from pOLE4/11 as the NcoI-XhoI-fragment, the region containing six His-coding sequence units fused to the GFP gene was excised from pHYB-MP-6×-GFP plasmid with XhoI and BamHI, and both DNA fragments were ligated into pNAP digested with NcoI and BamHI (FIG. 11).

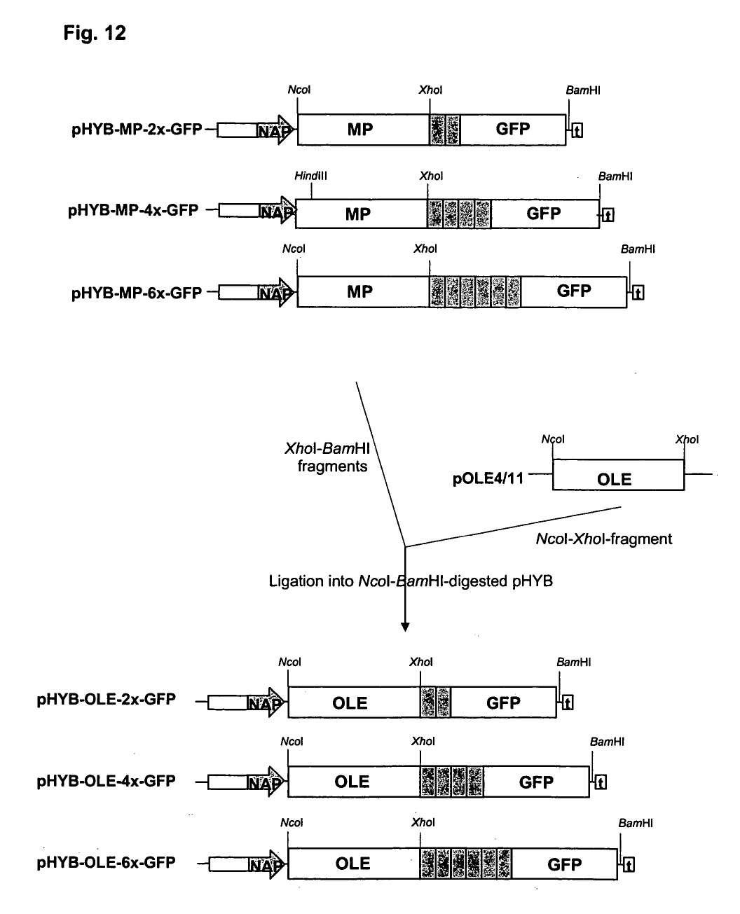

The set of expression vectors based on the hybrid promoter was constructed similarly. The oleosin gene was excised from pOLE4/11 as a NcoI-XhoI-fragment, the region containing two His-coding sequence units fused to the GFP gene was excised from pHYB-MP-2×-GFP with XhoI and BamHI, and both DNA fragments were ligated into pHYB digested with NcoI and BamHI, resulting in construct pHYB-OLE-2×-GFP. To obtain pHYB-OLE4×-GFP, the oleosin gene was excised from pOLE4/11 as a NcoI-XhoI-fragment, the region containing four His-coding sequence units fused to the GFP gene was excised from pHYB-MP-4×-GFP with XhoI and BamHI, and both DNA fragments were ligated into pHYB digested with NcoI and BamHI. To obtain pHYB-OLE-6×-GFP, the oleosin gene was excised from pOLE4/11 as a NcoI-XhoI-fragment, the region containing six His-coding sequence units fused to the GFP gene was excised from pHYB-MP-6×-GFP with XhoI and BamHI, and both DNA fragments were ligated into pHYB digested with NcoI and BamHI (FIG. 12).

### EXAMPLE 18

### Cloning of Cys/Met-enriched Oleosin and TMV 30K MP Genes into Expression Plasmids Containing the CaMV 35S, Napin, and Chimeric Promoters

Cys/Met-enriched sequences of three different lengths (with two, four, and six repeats of the Cys/Met-encoding sequence unit, example 12) were selected. The MP gene fused to two Cys/Met-coding units was excised from pMP-Cys/Met-2×with EcoRI and SalI, the GFP gene was excised from pRT-GFP as the XhoI-BamHI-fragment, and both DNA fragments were ligated into pRT101 digested with EcoRI and BamHI to give the construct pRT-MP-Cys/Met-2×-GFP. The MP gene fused to four Cys/Met-coding units was excised from pMP-Cys/Met-4× with EcoRI and SalI, the GFP gene was excised from pRT-GFP as a XhoI-BamHI-fragment, and both DNA fragments were ligated into pRT101 digested with EcoRI and BamHI to obtain pRT-MP-Cys/Met4×-GFP (FIG. 7).

To obtain equivalent constructs under the control of the napin promoter (replacing the CaMV 35S promoter), the N-terminal portion of the TMV MP gene was excised as a NcoI-HindIII-fragment from pGEM-30K; the fragment containing the rest of the MP gene fused to two Cys/Met-coding units and the GFP gene was excised from pRT-MP-Cys/Met-2×-GFP with HindIII and XbaI, and both fragments were ligated into pNAP digested with NcoI and BamHI to give pNAP-MP-Cys/Met-2×-GFP. In a similar fashion, to obtain pNAP-MP-Cys/Met4×-GFP, the N-terminal portion of the TMV 30K MP gene was excised as the NcoI-HindIII-fragment from pGEM-30K, and the fragment containing the rest of the MP gene fused to four Cys/Met-coding units and the GFP gene was excised from pRT-MP-Cys/Met-2×-GFP with HindIII and XbaI, and both fragments were ligated into pNAP digested with NcoI and BamHI. Finally, to construct pNAP-MP-Cys/Met-6×-GFP, the N-terminal portion of the TMV MP gene was excised as a NcoI-HindIII-fragment from pGEM-30K, and the fragment containing the rest of the MP gene fused to six Cys/Met-coding units and the GFP gene was excised from pRT-MP-Cys/Met-6×-GFP with HindIII and XbaI, and both fragments were ligated into pNAP digested with NcoI and BamHI (FIG. 8).

Similar cloning was undertaken to obtain the constructs in which the MP-Cys/Met-GFP fusion genes were placed under the control of the hybrid promoter (HYB). The N-terminal portion of the TMV MP gene was excised as a NcoI-HindIII-fragment from pGEM-30K, the fragment containing the rest of the MP gene fused to two Cys/Met-coding units and the GFP gene was excised from pRT-MP-Cys/Met-2×-GFP with HindIII and XbaI, and both fragments were ligated into pHYB digested with NcoI and BamHI to give pHYB-MP-Cys/Met-2×-GFP. To obtain pHYB-MP-Cys/Met-4×-GFP, the N-terminal portion of the TMV MP gene was excised as a NcoI-HindIII-fragment from pGEM-30K, the fragment containing the rest of the MP gene fused to four H is-coding units and the GFP gene was excised from pRT-MP-Cys/Met-4×-GFP with HindIII and XbaI, and both fragments were ligated into pHYB digested with NcoI and BamHI. To construct pHYB-MP-Cys/Met-6×-GFP, the N-terminal portion of the TMV MP gene was excised as a NcoI-HindIII-fragment from pGEM-3 OK, the fragment containing the rest of the MP gene fused t o six Cys/Met-coding units and the GFP gene was excised from pRT-MP-Cys/Met-6×-GFP with HindIII and XbaI, and both fragments were ligated into pHYB digested with NcoI and BamHI. Based on the oleosin gene from pOLE4/11, three sets of expression vectors were constructed that had the CaMV 35S, napin, or hybrid promoters, each set containing Cys/Met-coding regions of three different lengths, as in example 16 (FIG. 9).

For construction of expression vectors based on the CaMV 35S promoter, the oleosin gene was excised from pOLE4/11 as a NcoI-XhoI-fragment, the region containing two Cys/Met-coding sequence units fused to the GFP gene was excised from pHYB-MP-Cys/Met-2×-GFP with XhoI and BamHI, and both DNA fragments were ligated into pRT100 digested with NcoI and BamHI resulting in the construct pRT-OLE-Cys/Met-2×-GFP. To obtain pRT-OLE-Cys/Met-4×-GFP, the oleosin gene was excised from pOLE4/11 as a NcoI-XhoI-fragment, the region containing four Cys/Met-coding sequence units fused to the GFP gene was excised from pHYB-MP-Cys/Met-4×-GFP with XhoI and BamHI, and both DNA fragments were ligated into pRT100 digested with NcoI and BamHI. To obtain pRT-OLE-Cys/Met-6×-GFP, the oleosin gene was excised from pOLE4/11 as a NcoI-XhoI-fragment, the region containing six Cys/Met-coding sequence units fused to the GFP gene was excised from pHYB-MP-Cys/Met-6×-GFP with XhoI and BamHI, and then both DNA fragments were ligated into pRT100 digested with NcoI and BamHI (FIG. 10).

Similarly, for expression vectors based on the napin promoter, the oleosin gene was excised from pOLE4/11 as a NcoI-XhoI-fragment, the region containing two Cys/Met-coding sequence units fused to the GFP gene was excised from pHYB-MP-Cys/Met-2×-GFP with XhoI and BamHI, and both DNA fragments were ligated into pNAP digested with NcoI and BamHI, resulting in construct pNAP-OLE-Cys/Met-2×-GFP. To obtain pNAP-OLE-Cys/Met-4×-GFP, the oleosin gene was excised from pOLE4/11 as a NcoI-XhoI-fragment, the region containing four Cys/Met-coding sequence units fused to the GFP gene was excised from pHYB-MP-Cys/Met-4×-GFP plasmid with XhoI and BamHI, and both DNA fragments were ligated into pNAP digested with NcoI and BamHI. To obtain pNAP-OLE-Cys/Met-6×-GFP, the oleosin gene was excised from pOLE4/11 as the NcoI-XhoI-fragment, the region containing six Cys/Met-coding sequence units fused to the GFP gene was excised from pHYB-MP-Cys/Met-6×-GFP plasmid with XhoI and BamHI, and both DNA fragments were ligated into pNAP digested with NcoI and BamHI (FIG. 11).

The set of expression vectors based on the hybrid promoter was constructed in a similar manner. The oleosin gene was excised from pOLE4/11 as a NcoI-XhoI-fragment, and the region containing two Cys/Met-coding sequence units fused to the GFP gene was excised from pHYB-MP-Cys/Met-2×-GFP with XhoI and BamHI, and both DNA fragments were ligated into pHYB digested with NcoI and BamHI, resulting in construct pHYB-OLE-Cys/Met-2×-GFP. To obtain pHYB-OLE-Cys/Met-4×-GFP, the oleosin gene was excised from pOLE4/11 as a NcoI-XhoI-fragment, the region containing four Cys/Met-coding sequence units fused to the GFP gene was excised from pHYB-MP-Cys/Met4×-GFP with XhoI and BamHI, and both DNA fragments were ligated into pHYB digested with NcoI and BamHI. To obtain pHYB-OLE-Cys/Met-6×-GFP, the oleosin gene was excised from pOLE4/11 as a NcoI-XhoI-fragment, and the region containing six Cys/Met-coding sequence units fused to the GFP gene was excised from pHYB-MP-Cys/Met-6×-GFP with XhoI and BamHI. Both DNA fragments were ligated into pHYB digested with NcoI and BamHI (FIG. 12).

### EXAMPLE 19

### Cloning of Glycine-enriched TMV 30K MP and Oleosin Genes into Expression Plasmids Containing the CaMV 35S, Napin, and Chimeric Promoters

Glycine-enriched sequences of two different lengths (with two and four repeats of the Gly-encoding sequence units) (Example 13) were selected. The MP gene fused to two Gly-coding units was excised from pMP-Gly-2× with EcoRI and Sail, the GFP gene was excised from pRT-GFP (See FIG. 7 for details) as the XhoI-BamHI-fragment, and both DNA fragments were ligated into pRT101 digested with EcoRI and BamHI to give the construct pRT-MP-Gly-2×-GFP. The MP gene fused to four Gly-coding units was excised from pMP-Gly-4x with EcoRI and SalI, the GFP gene was excised from pRT-GFP as a XhoI-BamHI-fragment, and both DNA fragments were ligated into pRT101 digested with EcoRI and BamHI to obtain pRT-MP-Gly-4×-GFP (FIG. 7).

To obtain equivalent constructs under the control of the napin promoter (replacing the CaMV 35S promoter), the NcoI-HindIII-fragment of the TMV MP gene was excised from pGEM-30K (the fragment containing the rest of the MP gene fused to two Gly-coding units) and the GFP gene was excised from pRT-MP-Gly-2×-GFP with HindIII and XbaI, and both fragments were ligated into pNAP digested with NcoI and BamHI to give pNAP-MP-Gly-2×-GFP. To obtain pNAP-MP-Gly-4×-GFP, the N-terminal portion of the TMV 30K MP gene was excised as the NcoI-HindIII-fragment from pGEM-30K, and the fragment containing the rest of the MP gene fused to four Gly-coding units and the GFP gene was excised from pRT-MP-Gly-2×-GFP with HindIII and XbaI, and both fragments were ligated into pNAP digested with NcoI and BamHI (FIG. 8).

To obtain the hybrid promoter construct (HYB), the N-terminal portion of the TMV MP gene was excised as a NcoI-HindIII-fragment from pGEM-30K, and the GFP gene was excised from pRT-MP-Gly-2×-GFP with HindIII and XbaI, and both fragments were ligated into pHYB digested with NcoI and BamHI to give pHYB-MP-Gly-2×-GFP. To obtain pHYB-MP-Gly-4×-GFP, the N-terminal portion of the TMV MP gene was excised as a NcoI-HindIII-fragment from pGEM-30K, the fragment containing the rest of the MP gene fused to two glycine-coding units and the GFP gene was excised from pRT-MP-Gly-2×-GFP with HindIII and XbaI, and both fragments were ligated into pHYB digested with NcoI and BamHI (FIG. 9).

Based on the oleosin gene from pOLE4/11, three sets of expression vectors were constructed that had the CaMV 35S, napin, or hybrid promoters, each set containing Gly-coding regions of two different lengths, as in example 16. For construction of expression vectors based on the CaMV 35S promoter, the oleosin gene was excised from pOLE4/11 as a NcoI-XhoI-fragment, the region containing two Gly-coding sequence units fused to the GFP gene was excised from pHYB-MP-Gly-2×-GFP with XhoI and BamHI, and both DNA fragments were ligated into pRT100 digested with NcoI and BamHI resulting in the construct pRT-OLE-Gly-2×-GFP. To obtain pRT-OLE-Gly-4×-GFP, the oleosin gene was excised from pOLE4/11 as a NcoI-XhoI-fragment, the region containing four Gly-coding sequence units fused to the GFP gene was excised from pHYB-MP-Gly-2×-GFP with XhoI and BamHI, and both DNA fragments were ligated into pRT100 digested with NcoI and BamHI (FIG. 10).

Similarly, for expression vectors based on the napin promoter, the oleosin gene was excised from p OLE4/11 as a NcoI-XhoI-fragment, the region containing two Gly-coding sequence units fused to the GFP gene was excised from pHYB-MP-Gly-2×-GFP with XhoI and BamHI, and both DNA fragments were ligated into pNAP digested with NcoI and BamHI, resulting in construct pNAP-OLE-Gly-2×-GFP. To obtain pNAP-OLE-Gly-4×-GFP, the oleosin gene was excised from pOLE4/11 as a NcoI-XhoI-fragment, the region containing four Gly-coding sequence units fused to the GFP gene was excised from pHYB-MP-Gly-4×-GFP plasmid with XhoI and BamHI, and both DNA fragments were ligated into pNAP digested with NcoI and BamHI (FIG. 11).

The set of expression vectors based on the hybrid promoter was constructed in a similar manner. The oleosin gene was excised from pOLE4/11 as a NcoI-XhoI-fragment, and the region containing two Gly-coding sequence units fused to the GFP gene was excised from pHYB-MP-Gly-2×-GFP with XhoI and BamHI, and both DNA fragments were ligated into pHYB digested with NcoI and BamHI, resulting in construct pHYB-OLE-Gly-2×-GFP. To obtain pHYB-OLE-Gly-4×-GFP, the oleosin gene was excised from pOLE4/11 as a NcoI-XhoI-fragment, the region containing four Gly-coding sequence units fused to the GFP gene was excised from pHYB-MP-Gly-4×-GFP with XhoI and BamHI, and both DNA fragments were ligated into pHYB digested with NcoI and BamHI (FIG. 12).

### EXAMPLE 20

### Cloning of Lysine-enriched TMV 30K MP and Oleosin Genes into Expression Plasmids Containing the CaMV 35S, Napin, and Chimeric Promoters

Lysine-enriched sequences of two different lengths (with two and four repeats of the Lys-encoding sequence units) (Example 14) were selected. The MP gene fused to two Lys-coding units was excised from pMP-Lys-2× with EcoRI and SalI, the GFP gene was excised from pRT-GFP as the XhoI-BamHI-fragment, and both DNA fragments were ligated into pRT101 digested with EcoRI and BamHI to give the construct pRT-MP-Lys-2×-GFP. The MP gene fused to four Lys-coding units was excised from pMP-Lys-4x with EcoRI and SalI, the GFP gene was excised from pRT-GFP as a XhoI-BamHI-fragment, and both DNA fragments were ligated into pRT101 digested with EcoRI and BamHI to obtain pRT-MP-Lys-4×-GFP, as in examples 17-19 and in FIG. 7.

To obtain equivalent constructs under the control of the napin and hybrid (HYB) promoters (replacing the CaMV 35S promoter), NcoI-HindIII-fragment of TMV MP gene was excised from pGEM-30K (the fragment containing the rest of the MP gene fused to two Lys-coding units) and the GFP gene was excised from pRT-MP-Lys-2×-GFP with HindII and XbaI, and both fragments were ligated into pNAP or pHYB digested with NcoI and BamHI to give pNAP-MP-Lys-2×-GFP and pHYB-MP-Lys-2×-GFP, respectively. To obtain pNAP-MP-Lys-4×-GFP and pHYB-MP-Lys4×-GFP the N-terminal portion of the TMV 30K MP gene was excised as the NcoI-HindIII-fragment from pGEM-30K, and the fragment containing the rest of the MP gene fused to four Lys-coding units and the GFP gene was excised from pRT-MP-Lys-2×-GFP with HindIII and XbaI, and both fragments were ligated into pNAP and pHYB-digested with NcoI and BamHI, respectively (FIGS. 8 and 9).

Based on the oleosin gene from pOLE4/11, three sets of expression vectors were constructed that had the CaMV 35S, napin, or hybrid promoters, each set containing Lys-coding regions of two different lengths, as in example 19. For construction of expression vectors based on the CaMV 35S, napin or HYB promoters, the oleosin gene was excised from pOLE4/11 as a NcoI-XhoI-fragment, the region containing two Lys-coding sequence units fused to the GFP gene was excised from pHYB-MP-Lys-2×-GFP with XhoI and BamHI, and both DNA fragments were ligated into pRT100 digested with NcoI and BamHI resulting in the constructs pRT-OLE-Lys-2×-GFP. pNAP-OLE-Lys-2×-GFP and pHYB-OLE-Lys-2×-GFP. To obtain pRT-OLE-Lys-4×-GFP, pNAP-OLE-Lys-4×-GFP, and pHYB-OLE-Lys-4×-GFP, the oleosin gene was excised from pOLE4/11 as a NcoI-XhoI-fragment, the region containing four Lys-coding sequence units fused to the GFP gene was excised from pHYB-MP-Lys-2×-GFP with XhoI and BamHI, and both DNA fragments were ligated into pRT 100 digested with NcoI and BamHI.

### EXAMPLE 21

### Transient Expression Systems

A transient expression assay was used to ensure protein expression and that the expressed protein had intact biological function(s) as compared to the native unmodified protein, and was accumulating in the targeted organ. This was facilitated by visual observation of the reporter protein. Confocal laser scanning microscopy was particularly useful because it allowed early detection of transgenic plants expressing amino acid-enriched proteins in-frame with the nucleotide sequence encoding the reporter protein. Antibody-based assays (e.g. enzyme linked immuno absorbent assays) and direct amino acid analysis could be used as alternative systems to detect transgenic plants expressing amino acid-enriched proteins in-frame with the nucleotide sequence encoding the reporter protein.

### EXAMPLE 22

### Testing the Activity of Napin and Chimeric Promoters using Transient Expression of the GFP Gene in Plant Cells Transformed by Particle Bombardment

To analyze relative transcription levels provided by the napin promoter in pNAP, the hybrid promoter in pHYB, and the CaMV 35S promoter in pRT100, the coding region of the reporter protein, GFP, was cloned into these plasmids. For construction of GFP-containing clones, a gene for a red-shifted GFP mutant (S65T) was used. The GFP coding region was cloned as a NcoI-BamHI-fragment into similarly digested pRT100, pNAP and pHYB.

Epidermal cells of Nicotiana benthamiana transiently transformed using particle bombardment (Example 8) with pHYB-GFP (containing the GFP coding region under the control of the hybrid promoter) expressed GFP brightly. Observed GFP expression levels were comparable to the control, pRT-GFP, in which GFP expression was under the control of the CaMV 35S promoter. These experiments indicated that pHYB-GFP expression cassette is fully functional in living plant cells (FIG. 6 shows pRT-OLE-4×-GFP and pHYB-OLE-4×-GFP). pNAP-GFP expresses in early stages in seed embryos and such embryos have been detected to be fluorescing.

### EXAMPLE 23

### Cloning of Expression Cassettes into Binary Vectors and Transformation of Corresponding Binary Vectors into Agrobacterium

Gene cassettes containing GFP-tagged histidine, cysteine/methionine, glycine and lysine-enriched oleosin (ole) and TMV MP genes under control of CaMV 35S, napin and chimeric promoters from pRT-derivatives were cloned into the T-DNA-located HindIII site of the plant transformation binary vector pBin19 prior to Agrobacterium transformation.

Agrobacterium tumefaciens AGLI carrying the hyper-virulent, attenuated tumor-inducing plasmid pTi Bo542 (Lazo et al., 1991. Biotechnology 3, 963-967) were transformed by the freeze-thaw method of Holsters (Holsters et al., 1978. Mol.Gen.Genet. 163, 181-187) with some modifications. Agrobacteria were cultivated on LB-agar plates supplemented with 0,5 g l⁻¹ MgSO₄ for two days at 26° C., followed by. cultivation on liquid 2×YT medium supplemented with 50 μg/ml rifampicin to mid-exponential phase, then washed with 150 mM NaCl and resuspended in 20 mM CaCl₂ at ˜1010 cfu ml⁻¹ prior to transformation. Aliquots of cell suspension were frozen in liquid nitrogen after addition of glycerol to a final concentration of 15%. After the addition of 5 μg of DNA to cells, the cells were thawed and incubated on ice for 15 minutes, prior to heat-shock at 37° C. for 5 minutes. Cells were diluted with fresh 2×YT medium and cultured for 2 hours at 28° C. with vigorous aeration prior to being transferred to 1% agar plates containing 50 g μg ml⁻¹ rifampicin and 100 μg ml⁻¹ kanamycin. Kanamycin-resistant colonies were screened by PCR-analysis for the presence/absence of recombinant constructs.

### EXAMPLE 24

### Agrobacterium-mediated Transformation of Brassica campestris

Transformation of Brassica campestris plants was done essentially as described (Moloney et al., Plant Cell Reports 8, 238-272, 1989). Cotyledons were co-cultivated with appropriate Agrobacterium cell suspensions for 2-3 days on 1% agar with Medium I (Murashige Minimal Organics (MMO) with 3% sucrose, 4 mg/l BAP, 0.7% Phytoagar) at 22° C. Cotyledons were rinsed three times in distilled water prior to transfer to petri dishes containing Medium II (MMO with 4 mg/L BAP, 3% sucrose, 300 mg/l Ticarcillin (DUCHEFA) and 0.7% Phytoagar). After a further 7-10 days, explants were transferred to Medium III (MMO with 4 mg/L BAP, 3% sucrose, 300 mg/l Ticarcillin, 25 mg/l Kanamycin and 0.7% Phytoagar). After additional 2-3 weeks incubation explants with green calli or immature green shoots were transferred to fresh Medium III. For shoot formation, calli and immature green shoots were transfered to Medium IV (MMO with 3% sucrose, 300 mg/l Ticarcillin, 25 mg/l Kanamycin and 0.7% Phytoagar). Fully formed shoots were transferred to Medium V (MMO with 0.2 mg/l IBA, 3% sucrose, 300 mg/l Ticarcillin and 0.7% Phytoagar) for rooting. Once an established root system had formed, shoots with roots were removed from agar and transferred to moist potting soil, and grown in 16 h light/8h dark photoperiod at light intensity of 12.1 μmol m⁻² s⁻¹ at 22° C.

### EXAMPLE 25

### Avoiding Gene Silencing in Brassica campestris

When using Ole encoding nucleotide sequence derived from B. campestris, there is a risk of post-transscriptional gene silencing. The risk of inducing post-transcriptional gene silencing in a transgenic plant due to nucleotide sequence homology between the transgene and the endogenous gene when transforming B. campestris with an Ole gene, was successfully avoided in the present invention by using an Ole gene from Arabidopsis thaliana, and not that from B. campestris. The length and the extent of homology of one gene to another affects gene silencing. If an introduced gene shares 100% sequence homology with an endogenous gene then gene silencing is likely. Depending on which Brassica oleosin is compared with which Arabidopsis oleosin (currently in the NCBI data base) sequence homology at the DNA level ranges approximately from 74%-87%. Thus the degree of shared homologies at the DNA level are clearly different; given that transgenic plants were produced that did not appear to undergo gene silencing. Third generation plants which had stable expresssion of 4×His -cassette (based on antibody detection and dot blot) were screened.

### EXAMPLE 26

### Expression of Constructs with Various Lengths of Histidine, Cysteine/methionine, Glycine and Lysine in Tobacco Epidermal Cells

The lengths of the engineered poly-amino acid sequences were thought to be crucial for the expression of the His, Cys/Met, Gly and Lys-constructs, for two reasons including stability and inherent limitations in the production capacity of the cells.

Long polyamino acid sequences, for example in the 8×His-series were unstable in an in vitro translation system (TNT® T7/SP6 Coupled Wheat Germ Extract System L5030, Promega corporation) and the 6×His-series restricted the growth of E. coli in liquid medium as shown in example Example 11. The inherent limitation of the cells to produce large a mounts of polyamino acids without having an effect on other proteins and their synthesis was also cruscial for the expression.

Therefore, all CaMV 35S promoter-driven constructs were tested in planta to examine the limitation(s) of the large polyamino acid sequences in plant cells. Despite the suspected limitation(s), all constructs (as prepared in examples 11-14) were expressed in tobacco epidermal cells. However, constructs containing longer polyamino acid sequences were relatively less well expressed than shorter polyamino acid sequences, therefore, constructs with four His, Cys/Met, Gly and Lys-cassettes were chosen for stable transformation experiments.

Western blot analysis of several plant lines (Table 1) showed that antibodies raised against histidine sequences, MP, oleosin or GFP (FIGS. 13-15) could be used to detect proteins with known molecular weights (MWs). The predicted MWs of oleosin, MP, GFP and 4× His-cassette (with 14 His-codons in each cassette) are 18.5, 30, 30, and 2.5 kDa, respectively. The appropriate MWs of fusion proteins can be calculated from these values as the sum of the MWs of each of the fusion components combined. These fusion proteins were shown to be identical in size to their predicted molecular weight, when examined with SDS-PAGE gel electrophoresis and by Western blot analysis. Stability of the transgene-encoded protein products (oleosin-His and MP-His) was investigated by Western blot analysis. Analysis of (selfed) plant generations (3^{rd} generation) after transformation (Table 1) showed clearly that transgene expression was stable over successive generations. Moreover, Western blot analysis of those transformed plants that were examined further revealed that the size of protein product remained constant regardless of the plant generation. These analyses also showed that the amount of fusion protein product was relatively constant between plant generations, indicative of an acquired stable expression level relative to other plant proteins.

### EXAMPLE 27

### The in Planta Production of Oleosin-histidine and MP-histidine Fusion Proteins

Four series of transgenic Brassica campestris lines were analysed (codes: 5, 8, 14 and 17). These series contained the following constructs: 5-series: pNAP-MP4×-His-GFP; 8-series: pHYB-MP-4×-His-GFP; 14-series: pNAP-OLE-4×-His-GFP; 17-series: pHYB-OLE4×-His-GFP.

Western blot analysis (Sambrook et al., Molecular cloning: A laboratory manual, 2^{nd} edn., Cold Spring Harbor, NY 1989) of several plant lines (Table 1) showed that antibodies (raised against His sequences, MP, oleosin or GFP (FIGS. 13-15) could be used to detect proteins with known molecular weights (MWs). MP, oleosin and GFP antibodies originated from Atabekov's laboratory. Poly-His antibody was commercial (Pierce, Rocford US). The predicted MWs of oleosin, MP, GFP and 4×His-cassette (with 14 His-codons in each cassette) were 18.5, 30, 30, and 2.5 kDa, respectively. The appropriate MWs of fusion proteins can be calculated from these values as the sum of the MWs of each of the fusion components combined i.e. MW of OLE-4×His-GFP is 62.5.

These fusion proteins OLE-4×His-GFP and MP-4×His-GFP were shown to be identical in size to the predicted molecular weight, when examined with SDS-PAGE gel electrophoresis and by Western blot analysis. Stability of the transgene-encoded protein products (oleosin-His and MP-His) was investigated by Western blot analysis. Analysis of (selfed) plant generations (3^{rd} generation) after transformation (Table 1) showed clearly that transgene expression was stable over successive generations. Moreover, Western blot analysis of examined transformed plants further revealed that the size of protein product remained constant regardless of the plant generation. These analyses also showed that the amount of fusion protein product was relatively constant between plant generations indicative of an acquired stable expression level relative to other plant proteins.

When His, MP and OLE antibodies were used both MP or OLE, and GFP were detected in the appropriate plant lines. This shows that the expression is stable and it is unlikely that His sequences would be “degraded out” if the carrier is present and expressed.

Table 1. Response of transgenic Brassica campestris plant lines to specific antibodies generated against the protein products encoded by the appropriate transgenes. Polyclonal OLE prepared against C-terminal portion of the protein and poly-MP generated against bacterially produced MP were generated in Atabekov's laboratory. Anti-His is commercially available from Pierce, Rocford US.

| | | Antibody response | | |
|---|---|---|---|---|
| Transgenic Plant line | Fusion protein | Oleosin | His | MP |
| 5.1A11 | MP | ND | +++ | +++ |
| 5.1A18 | MP | ND | +++ | +++ |
| 8.26 | MP | ND | + | + |
| 8.29 | MP | ND | + | + |
| 14.17C2 | Oleosin | ++ | ++ | ND |
| 14.5C8 | Oleosin | + | + | ND |
| 14.12A10 | Oleosin | +++ | +++ | ND |

ND = not detected,
+ = weak response,
++ = good response
and +++ = strong response.
Transgenic plant lines of 5 and 14 series contained NAP promoter and transgenic plant lines of 8 and 17 series contained HYB promoter.

### EXAMPLE 28

### Amino Acid Analysis of Seeds from Transgenic Brassica campestris Plant Lines Expressing Four Histidine Cassettes

Replicate samples of seeds destined for amino acid analysis were obtained from transgenic Brassica lines (T2) expressing four histidine cassettes (lines 5 and 8 have MP as a carrier, lines 14 and 17 have OLE as a carrier; see FIG. 3 and Example 27). Amino acid analyses were carried out on the total protein sample obtained from seeds (Table 2). The analysis was performed in Animal Nutrition Section in Agricultural Research Centre, Jokioinen, Finland according to European Commission Directive 98/64/EC. (1998).

TABLE 2
Seeds used for amino acid analysis.

| Plant line | Amount of seeds | Seed weight (mg) | Protein weight (mg) |
|---|---|---|---|
| WT | 10 | 33 | 11.3 |
| 14.5C8 | 8 | 11.5 | 7.2 |
| 14.12A10 | 9 | 30.9 | 19.4 |

WT = wild type

Transgenic Brassica campestris plant line 14.12A10 contained 30% more histidine compared to wild-type plant (Table 3 ). The increased amount of histidine did not a ccumulate at the expense of other amino acids, but altered the amount of total proteins.

TABLE 3
Relative increase of the histidine in
transgenic B. campestris seeds.

| | Amount | |
|---|---|---|
| Plant line | of histidine (g) per protein (kg) | Increase of histidine % |
| WT | 3.20 | 0 |
| 14.5C8 | 3.20 | 0 |
| 14.12A10 | 4.74 | 32 |

WT = wild type

In another analysis four transgenic lines (8.23, 8.6A1, 14.121) and 17.20C20 and one wild type line (designated WT) were used. These lines correspond to plants transformed with an 8-series, or 14-series or 17-series histidine cassette (See Example 27). The amount of histidine is expressed as g (of histidine)/Kg (total protein) in Table 4.

TABLE 4
The amount of histidine in Brassica campestris seeds.

| | Amount of histidine (g) per |
|---|---|
| Plant line | protein (kg) |
| WT | 6.95 |
| 8.23 | 4.92 |
| 8.6A1 | 7.19 |
| 14.121 | 7.37 |
| 17.20C20 | 6.76 |

WT = wild type

The increase of histidine was between 3-9% when the increase of other amino acid levels were excluded.

We claim:

1. A method for increasing the content of one or more selected amino acids in a selected tissue or organ of a plant comprising transformation of a plant with at least one recombinant nucleotide sequence construct comprising tissue or organ specific regulatory sequences driving transcription during selected stages of morphogenesis, the construct being operably linked to a chimeric nucleotide sequence comprising:
(i) a nucleotide sequence encoding a carrier protein comprising a plant specific protein enabling targeted expression of an amino acid enriched protein in the selected tissue or organ of the plant; said nucleotide sequence lacking a termination codon and being fused in frame with
(ii) at least one nucleotide sequence comprising a selected optimal number of codons encoding an amino acid sequence comprising a selected combination of one or more amino acid residues, wherein said optimal number of codons is selected by determining the number allowing a stable translation;
said recombinant nucleotide sequence construct enabling stable targeted expression of the selected amino acid enriched carrier protein having a stable polyamino acid extension in the selected tissue or organ of the plant.

2. The method according to claim 1, wherein the construct with the optimal number of codons allowing stable translation of the polyamino acid extension is selected using a cell free in vitro translation (IVT) system.

3. The method according to claim 1, wherein the targeted expression in the selected tissue or organ of the plant is enabled by a carrier protein having a stable polyamino acid extension and intact biological functions as compared to a corresponding unmodified carrier protein.

4. The method according to claim 1, wherein the construct enabling stable targeted expression of the carrier protein having a polyamino acid extension is selected with a transient expression assay, comprising a nucleotide sequence encoding a reporter protein fused in frame with the construct according to claim 1.

5. The method according to claim 1, wherein the construct enabling stable targeted expression of the carrier protein having a polyamino acid extension is selected by detecting the expression of a reporter protein in a plant cell.

6. The method according to claim 1, wherein the construct enabling a stable targeted expression of the carrier protein having a polyamino acid extension in a selected tissue or organ of a plant is obtainable by a method comprising the steps:
(a) fusing the recombinant nucleotide sequence construct of claim 1 in frame with a nucleotide sequence encoding a reporter protein;
(b) selecting in a cell free translation (IVT) system a construct with an optimal number of codons allowing stable translation of a polyamino acid extension;
(c) introducing the construct obtained in step (b) into a plant cell;
(d) selecting with a transient expression assay constructs enabling stable targeted expression of the carrier protein having a polyamino acid extension by detecting the expression of the reporter protein in a plant cell;
(e) removing the nucleotide sequence encoding the reporter gene from the constructs selected in step (d) to obtain a construct lacking the nucleotide sequence encoding the reporter protein;
(f) transforming a plant with the construct obtained in step (e).

7. The method according to claim 6, wherein the construct obtained in step (b) and comprising a nucleotide sequence encoding a reporter protein is introduced into a plant cell by microprojectile bombardment.

8. The method according to claim 6, wherein the construct providing stable targeted expression of the reporter protein is selected and subsequent to removal of the nucleotide sequence encoding the reporter protein is introduced into plants using an Agrobacterium mediated transformation.

9. The method according to claim 1, wherein the codons encoding the polyamino acid extension comprises histidine, cysteine, methionine, glycine, lysine, tryptophan, alanine, valine, leucine, isoleucine, proline, phenylalanine, tyrosine, serine, threonine, arginine, aspartate, glutamate, asparagines, glutamine encoding codons or any combination thereof.

10. The method according to claim 1, wherein the number of codons encoding the selected polyamino acid extension is from four to eighty.

11. The method according to claim 1, wherein the selected tissue or organ of a plant is a seed.

12. The method according to claim 1, wherein the selected tissue or organ of the plant is a cell wall or a cell membrane.

13. The method according to claim 1, wherein the selected tissue or organ of the plant is an oil body.

14. The method according to claim 1, wherein the plant specific carrier protein enabling stable targeted expression in a plant tissue or organ is a protein functioning in the intracellular trafficking pathway of the plant.

15. The method according to claim 14, wherein the carrier protein is a cell wall protein or plant viral protein.

16. The method according to claim 15, wherein the carrier protein comprises oleosin, caleosin, steroleosin, cruciferin, napin or a plant viral movement protein.

17. The method according to claim 16, wherein the carrier protein is a movement protein of Tobacco mosaic virus (TMV MP).

18. The method according to claim 1, wherein the regulatory sequence is a promoter expressing during embryogenesis.

19. The method according to claim 1, wherein the regulatory sequence comprises a napin (NAP), a Cauliflower mosaic virus 35S (35S), a chimeric hybrid (HYB), a 19S, a nopalin, a phaseolin, a steroleosin, a caleosin, a cruciferin, an Alfalfa mosaic virus (AMV), a heat-shock, an albumin 2S or an oleosin promoter.

20. The method according to claim 19, wherein the napin (NAP) promoter is a napin (NAP) promoter of Arabidopsis thaliana.

21. The method according to claim 19, wherein the hybrid (HYB) promoter is a chimeric promoter comprising an enhancer sequence of the CaMV 35S promoter and the entire napin (NAP) promoter.

22. The method according to claim 1, wherein stable targeted expression of the carrier protein having a polyamino acid extension is provided by using a movement protein expressing under the control of a napin (NAP) promoter.

23. The method according to claim 6, wherein the reporter protein is a nucleotide sequence encoding a detectable protein.

24. The method according to claim 6, wherein the reporter protein is a fluorescent protein.

25. The method according to claim 6, wherein the reporter protein is a green fluorescent protein (GFP), a red fluorescent protein, a β-glucuronidase, an obelin or a luciferase.

26. A recombinant nucleotide sequence construct for increasing the content of one or more selected amino acids in a selected tissue or organ of a plant, wherein the construct which enables stable targeted expression of an amino acid-enriched protein having a polyamino acid extension in a selected tissue or organ of a plant comprises a tissue or organ specific regulatory sequence driving transcription during selected stages of morphogenesis operably linked to a chimeric nucleotide sequence comprising;
(a) a nucleotide sequence encoding a carrier protein comprising a plant specific protein enabling stable targeted expression in a selected tissue or organ of a plant; said nucleotide sequence lacking a termination codon and being fused in frame with
(ii) at least one nucleotide sequence comprising a selected optimal number of codons allowing stable translation of the polyamino acid extension, and encoding an amino acid sequence comprising a selected combination of one or more amino acid residues.

27. The construct according to claim 26, wherein the construct, which enables stable targeted expression in the selected tissue or organ of the plant comprises a nucleotide sequence encoding a carrier protein having a polyamino acid extension and intact biological functions as compared to the corresponding unmodified carrier protein.

28. The construct according to claim 26, wherein the construct enabling the selection of a construct allowing targeted expression of the carrier protein having a polyamino acid extension comprises a nucleotide sequence encoding a reporter protein fused in frame with the chimeric nucleotide sequence according to claim 26.

29. The construct according to claim 26, wherein the plant specific carrier protein enabling stable targeted accumulation in a plant tissue or organ comprises a protein functioning in the secretory intracellular trafficking pathway.

30. The construct according to claim 26, wherein the carrier protein is a plant cell wall protein or plant viral protein.

31. The construct according to claim 26, wherein the carrier protein is oleosin, caleosin, steroleosin, cruciferin, napin or a plant viral movement protein.

32. The construct according to claim 26, wherein the carrier protein is a movement protein of Tobacco mosaic virus (TMV MP).

33. The construct according to claim 26, wherein the codons comprise histidine, cysteine, methionine, glycine, lysine, tryptophan, alanine, valine, leucine, isoleucine, proline, phenylalanine, tyrosine, serine, threonine, arginine, aspartate, glutamate, asparagines, glutamine encoding codons or any combination thereof.

34. The construct according to claim 26, wherein the optimal number of codons is from 4 to eighty.

35. The construct according to claim 26, wherein the regulatory sequence is a promoter expressing during embryogenesis.

36. The construct according to claim 26, wherein the regulatory sequence comprises a napin (NAP), a Cauliflower mosaic virus (CMV 35S), a chimeric hybrid (HYB), a19S, a nopalin, a phaseolin, a steroleosin, a caleosin, a cruciferin, an Alfalfa mosaic virus (AMV), a heat-shock, an albumin 2S or an oleosin promoter.

37. The construct according to claim 26, wherein the promoter is a napin (NAP) promoter of Arabidopsis thaliana.

38. The construct according to claim 36, wherein the chimeric hybrid (HYB) promoter comprises an enhancer sequence of the CaMV 35S promoter and the entire napin (NAP) promoter.

39. The construct according to claim 28, wherein the reporter protein is a detectable protein.

40. The construct according to claim 28, wherein the reporter protein is a fluorescent protein.

41. The construct according to claim 28, wherein the reporter protein is green fluorescent protein (GFP), red fluorescent protein, β-glucuronidase, obelin or luciferase.

42. The construct according to claim 28, wherein the construct comprises a nucleic acid sequence encoding a carrier protein having a histidine enriched extension and a green fluorescent protein (GFP).

43. The method according to claim 1 for producing a composition comprising in plant material an amino acid-enriched carrier protein having a polyamino acid extension, wherein the content of amino acids in the plant material obtained by the method as compared to the amino acid content in a corresponding unmodified wild type plant is at least 2:1.

44. The method according to claim 43 for producing an amino acid-enriched feed of an oil cake obtained after recovery of oil from plants.

45. The construct according to claim 26 for producing a composition comprising in plant material an amino acid-enriched carrier protein having a polyamino acid extension, wherein the content of amino acids in the plant material obtained by the method as compared to the amino acid content in a corresponding unmodified wild type plant is at least 2:1.

46. A plant transformed with the construct according to claim 26.

47. A plant transformed with the construct according to claim 28.

48. A plant cell transformed with the construct according to claim 26.

49. A plant cell transformed with the construct according to claim 28.

50. A plant cell-line transformed with the construct according to claim 26.

51. A plant cell-line transformed with the construct according to claim 28.

52. A composition obtainable by the method according to claim 1, which composition comprises in plant material an amino acid-enriched carrier protein having a polyamino extension, in which composition the content of amino acids in the plant material obtained by the method as compared to the amino acid content in a corresponding unmodified wild type plant is at least 2:1.


SEQUENCE LISTING (17 sequences)

SEQ ID NO: 1 - DNA, 45 bp, Artificial Sequence
  Description of Artificial Sequence synthetic
gcgcctcgagttcaccatcaccatcaccatcacgggcaccatcac

SEQ ID NO: 2 - DNA, 17 bp, Artificial Sequence
  Description of Artificial Sequence synthetic
catcaccatcaccatgg

SEQ ID NO: 3 - DNA, 45 bp, Artificial Sequence
  Description of Artificial Sequence synthetic
ccggatcctaaagtcgaccatggtgatggtgatggtgatggtgcc

SEQ ID NO: 4 - DNA, 50 bp, Artificial Sequence
  Description of Artificial Sequence synthetic
cacctcgagtatgttgttgcatgtgcatgtgctgttgcatgtcgacaaac

SEQ ID NO: 5 - DNA, 50 bp, Artificial Sequence
  Description of Artificial Sequence synthetic
gtttgtcgacatgcaacagcacatgcacatgcaacaacatactcgaggtg

SEQ ID NO: 6 - DNA, 57 bp, Artificial Sequence
  Description of Artificial Sequence synthetic
gcgcctcgagttggtggaggtggaggcggtggaggtggcgtcgacaaatggatcccc

SEQ ID NO: 7 - DNA, 60 bp, Artificial Sequence
  Description of Artificial Sequence synthetic
ggggatccatttgtagacgccacctcctccaccgcctccacctccaccaactcgaggcgc

SEQ ID NO: 8 - DNA, 66 bp, Artificial Sequence
  Description of Artificial Sequence synthetic
gcgcctcgagttaaaaagaaaaagaaaaagaaaaagaaaaagaaaaaggtcgacaaatggatcccc

SEQ ID NO: 9 - DNA, 66 bp, Artificial Sequence
  Description of Artificial Sequence synthetic
ggggatccatttgtcgacctttttctttttctttttctttttctttttctttttaactcgaggcgc

SEQ ID NO: 10 - DNA, 34 bp, Artificial Sequence
  Description of Artificial Sequence synthetic
aaaaccatggcggatacagctagaggaacccatc

SEQ ID NO: 11 - DNA, 37 bp, Artificial Sequence
  Description of Artificial Sequence synthetic
ggggccatgggagtagtgtgctggccaccacgagtac

SEQ ID NO: 12 - DNA, 31 bp, Artificial Sequence
  Description of Artificial Sequence synthetic
gcgcctcgagaagtagtgtgctggccaccac

SEQ ID NO: 13 - DNA, 31 bp, Artificial Sequence
  Description of Artificial Sequence synthetic
gcggaattcccatggctctagttgttaaagg

SEQ ID NO: 14 - DNA, 32 bp, Artificial Sequence
  Description of Artificial Sequence synthetic
agacctcgaggaaacgaatccgattcggcgac

SEQ ID NO: 15 - DNA, 31 bp, Artificial Sequence
  Description of Artificial Sequence synthetic
tgtgggatcctacgcaacgggagagcaccca

SEQ ID NO: 16 - DNA, 27 bp, Artificial Sequence
  Description of Artificial Sequence synthetic
cttgttagccatggtttgctatttgtg

SEQ ID NO: 17 - DNA, 26 bp, Artificial Sequence
  Description of Artificial Sequence synthetic
tcttactcgagtgaaaccaaattaac